(19) United States
(12) Patent Application Publication
(10) Pub. No.: US 2011/0243853 A1
(43) Pub. Date: Oct. 6, 2011

(54) MODELS OF ERYTHROPOIESIS

(76) Inventors: Catriona Helen M. Jamieson, LaJolla, CA (US); Ken Kaushansky, Del Mar, CA (US); Charlene Barroga, San Diego, CA (US); Ifat Geron, San Diego, CA (US); Annelie Schairer, San Diego, CA (US); Edward Kavalerchik, San Diego, CA (US)

(21) Appl. No.: 12/446,203

(22) PCT Filed: Oct. 24, 2007
(86) PCT No.: PCT/US07/22600

§ 371 Date: Feb. 2, 2011

Related U.S. Application Data
(60) Provisional application No. 60/854,488, filed on Oct. 25, 2006.

(51) Int. Cl. A01K 67/033; A61K 49/00; C12Q 1/02

(52) U.S. Cl. 424/9.2; 800/9; 435/29

(57) ABSTRACT

Non human animal models are provided for diseases involving erythroid function, particularly myeloproliferative disease. The models are useful for testing and screening of biologically active agents that affect erythropoiesis, and erythroid function. In the animal models of the invention, a hematopoietic stem or progenitor cell (HSC) population that has been genetically altered by the introduction of a mutant JAK2 coding sequence is transplanted into an immunocompromised, xenogeneic, non-human recipient. The recipient animal is engrafted with the cell population at a high frequency, and develops a myeloproliferative disorder characterized by polycythemia.

### BACKGROUND OF THE INVENTION

Myeloproliferative disorders (MPDs), such as polycythemia vera (PV), essential thrombocythemia (ET) and myelofibrosis (MF) are clonal hematopoietic disorders typified by overproduction of terminally differentiated cells in part as a result of hypersensitivity of marrow progenitor cells to hematopoietic growth factors. Clonal derivation of MPDs from a primitive hematopoietic progenitor was first suggested by studies demonstrating the same glucose-6-phosphate dehydrogenase (G6PD) allele in PV erythrocytes, granulocytes and platelets and later confirmed by phosphoglycerate kinase gene PCR that revealed clonal involvement of the myeloid and erythroid lineages in female PV patients.

Several recent reports provided critical insight into the molecular events involved in the development of PV by identifying a mutation (V617F) that constitutively activates the JAK2 tyrosine kinase in the majority of patients with PV and approximately 50% of patients with essential thrombocythemia (ET) and idiopathic myelofibrosis (MF) (Baxter et al. (2005). Lancet. 365, 1054-1061). Mutant JAK2 gene expression in the Ba/F3 growth factor dependent cell line resulted in erythropoietin (EPO) hypersensitivity and growth factor-independent survival (James et al., 2005). Moreover, transplantation of mouse marrow cells transduced with the JAK2 V617F mutant allele into lethally irradiated recipients resulted in erythrocytosis typical of PV (James et al. (2005) Nature. 434, 1144-1148). In addition, mice transplanted with JAK2 V617F-expressing cells developed a PV-like disease that progressed to myelofibrosis in manner analogous to human PV (Wernig et al. (2006) Blood 107, 4274-4281). These studies support a critical role for JAK2 V617F in the pathogenesis of a large proportion of MPDs (Kaushansky (2005) Blood 105, 4187-4190).

Although JAK2 V617F-driven MPDs such as PV, ET and MF have a combined incidence that is five-fold higher than chronic myeloid leukemia (CML), the first cancer to be associated with a pathognomic molecular abnormality at the hematopoietic stem cell and the first cancer to be treated with molecularly targeted therapy, there has been no treatment developed to date that selectively inhibits JAK2 kinase activity. While JAK2 V617F⁺ PV and ET have a lower rate of progression to acute leukemia than CML, both quality and quantity of life are detrimentally affected by a high prevalence of major thrombotic events.

In addition, primary myelofibrosis and AML or myelofibrosis that develop following sustained myeloproliferation in JAK2 V617F⁺ PV or ET are relatively recalcitrant to current forms of treatment thereby, providing the impetus for developing selective JAK2 inhibitors (Tefferi et al. (2006) Blood. 108, 1158-1164; Harrison et al. (2005) N. Engl. J. Med. 353, 33-45).

The design and screening of effective therapies for erythroid diseases is made complicated by the lack of effective models for drug screening. Humans cannot intentionally be studied in the pre-clinical phase, red blood cells cannot be maintained in continuous culture, and animal erythropoiesis may differ from human in important features. As a result, there is a lack of reliable information on which to base decisions about clinical trials.

In addition to erythroid diseases such as polycythemia vera, malaria remains one of the most deadly infectious diseases and there is a clear need to develop novel control strategies to limit the parasite in people. Development treatment for Plasmodium falciparum at the preclinical stage has been hampered by a lack of a good in vivo challenge model.

With drug discovery moving from target identification to validations, reliable biological systems are necessary to confirm, validate and support the recent explosion in the number of potential new drug targets and drug compounds. The development of robust, reproducible and scaleable animal models that physiologically resemble human disease is very desirable; i.e. models erythroid disease can be used as treatment models and not only preventive ones. Such animal models must posses the utility to rapidly advance experimental drug leads rapidly and reliably in a semi- to high through-put fashion, leading to novel, effective and safe therapeutics.

### PUBLICATIONS

Jamieson et al. (2006) Proc. Natl. Acad. Sci. USA. 103, 6224-6229; Kennedy et al. (2006) Proc. Natl. Acad. Sci. USA. 103, 16930-16935.

### SUMMARY OF THE INVENTION

Models are provided for diseases involving erythroid function, particularly myeloproliferative disease, e.g. polycythemia vera (PV). The models are useful for testing and screening of biologically active agents that affect erythropoiesis, and erythroid function. In the animal models of the invention, a hematopoietic stem or progenitor cell (HSC) population, e.g. HSC population, that has been genetically altered by the introduction of a mutant JAK2 coding sequence is transplanted into an immunocompromised, xenogeneic, non-human recipient, e.g. a rodent. The genetically altered hematopoietic cell population may further comprise a bioluminescent label. The recipient animal is engrafted with the cell population at a high frequency, and develops a myeloproliferative disorder characterized by polycythemia, which can provide a model for the human disease PV. The animals provide a useful model for erythropoiesis, for drug/gene screening in the prevention and treatment of erythroid disease in humans, and for diseases that affect erythrocytes, e.g. malaria, etc.

A specific mutation in a pseudo-kinase region of human Jak-2, V617F, leads to constitutive JAK2 expression. This mutation is observed to skew differentiation of human stem cells (HSC) and stem cell progenitors toward the erythroid lineage. Such alteration of hematopoiesis can be inhibited with a selective JAK2 inhibitor. The methods and animal models of the invention provide a means of using the specific V617F mutation to evaluate agonists and antagonists of hematopoietic cell growth and differentiation, particularly erythroid and megakaryocytic growth and differentiation, as well as to test novel agents directed at pathogens that affect specific hematopoietic lineages, such as malaria. I

The ability of JAK2 V617F to alter human hematopoietic progenitor cell fate decisions is shown both in vitro and in a xenogeneic transplantation model of human PV. Erythroid differentiation potential in the presence or absence of a selective JAK2 inhibitor, TG101348, is also provided, and demonstrates the ability of the animal model of the invention to pre-clinically assess the efficacy of a candidate agent as therapy for JAK2-driven myeloproliferative disorders.

### BRIEF DESCRIPTION OF THE DRAWINGS

FIGS. 1A-1D. Inhibition of PV Progenitor Erythroid Differentiation by TG101348. A. Structure of TG101348. TG101348 is a small molecule ATP-competitive inhibitor designed and synthesized at TargeGen using structure based drug design methods to inhibit JAK2 (IC50=3 nM), but not other closely related kinases (e.g., JAK3 IC50=1040 nM). B. In four experiments, HSC(CD34⁺CD38⁻CD90⁺Lin⁻), progenitors (CD34⁺CD38⁺Lin⁻) or common myeloid progenitor (CMP; CD34⁺CD38⁺CD123⁺CD45RA⁻Lin⁻) cells from separate JAK2 V617F⁺ PV patients were clone-sorted (25 cells/well) with the aid of a FACS Aria and treated with 0, 30, 100, 300 or 600 nM of TG101348 in human cytokine supplemented methylcellulose. Differential colony counts were performed on day 14. Differential PV HSC and progenitor colony counts from peripheral blood donated by patients 3 and 4 are depicted in the presence of increasing concentrations of TG101348. C. Erythroid colony formation was readily inhibited by TG101348. Left Panel: Representative photomicrographs (50× magnification) of PV progenitor colonies treated with 0, 30 and 100 nM and the corresponding JAK2 mutant allele frequency (% V617F) determined by sequencing on pooled colonies. Right Panel: Progenitor (CD34⁺CD38⁺Lin⁻) cells from JAK2 V617F⁺ PV blood were clone-sorted (25 cells/well; n=4 wells+/−S.E.M.) with the aid of a FACS Aria and treated with 0, 30, 100, 300 or 600 nM of TG101348 in human cytokine supplemented methylcellulose. Differential colony counts were performed on day 14. There was a significant reduction in PV progenitor erythroid colony formation following treatment with 300 nM (p=0.02) or 600 nM (p=0.02) of TG101348. D. In three experiments, pooled HSC colonies were subjected to JAK2 V617F sequencing analysis. In PV patient sample 4 (PV4), pooled HSC colonies had a mutant allele frequency of 90% whereas no variant was detected in the TG101348 (300 nM) treated colonies. However, in another patient sample (PV5) the mutant allele frequency did not decrease as dramatically following treatment suggesting some individual variation in sensitivity to JAK2 inhibition.

FIGS. 2A-2C. Inhibition of JAK2 V617F Driven Erythroid Differentiation with TG101348. A. Photomicrographs (Zeiss Axiovert, 50× magnification) of normal cord blood hematopoietic stem cell (HSC) day 14 colonies with no vector, backbone vector, JAK2 V617F (Mutant JAK2) or wild-type JAK2 (WT JAK2) demonstrated that mutant JAK2 gave rise to a preponderance of erythroid (BFU-E) colonies while WT JAK2 induced more mixed (CFU-GEMM) colony formation than backbone vector controls (n=4 experiments). B. Human cord blood HSC derived colonies were collected after 14 days in methylcellulose culture and lentiviral JAK2 (mJAK2) PCR was used to verify transduction with the lentiviral vectors. In addition, bands were extracted and sequenced to verify presence of WTJAK2 and mutant JAK2 V617F. C. Human cord blood HSC(CD34⁺CD38⁻CD90⁺Lin⁻) transduced with lentiviral backbone, JAK2 V617F (mutant JAK2) or wild-type JAK2 (WT JAK2) vectors, (25 cells/well in 96 well plate with human cytokine supplemented methylcellulose) were treated with (+) or without (−) 300 nM of TG101348, a selective JAK2 inhibitor, and colonies were scored on day 14. Non-transduced HSC served as a control. These experiments (n=4) demonstrated inhibition of mutant JAK2 skewed erythroid colony formation with TG101348, a selective JAK2 inhibitor.

FIGS. 3A-3D. Enhanced PV Progenitor Erythroid Engraftment is Inhibited by TG101348. A. Schema of Bioluminescent Engraftment Analysis of Human Progenitors in RAG2^{-/-}γ_{c}^{-/-} Mice. B. In 4 experiments, CD34-enriched or FACS-purified hematopoietic stem cells (HSC) and progenitors (CD34⁺CD38⁺) from PV patient blood or normal human cord blood were marked with Luc-GFP lentivirus and 48 hrs later transplanted intrahepatically in newborn immunodeficient mice. Mice were analyzed for bioluminescence with the aid of a non-invasive in vivo imaging system (IVIS 200, Caliper Inc). Mice were sacrificed 6 weeks after transplantation. Hematopoietic organs (spleen, liver, bone marrow and thymus) were harvested and human hematopoietic engraftment analyzed by FACS. This analysis revealed that PV HSC and progenitors gave rise to enhanced human erythroid engraftment when compared with their normal counterparts. C. FACS purified hematopoietic stem cells (HSC) and progenitors (CD34⁺CD38⁺Lin⁻) from polycythemia vera (PV) patient blood or normal cord blood were lentivirally transduced with luciferase-GFP for 48 hrs followed by intrahepatic transplantation into neonatal immunodeficient (RAG2^{-/-}γ_{c}^{-/-}) mice. Bioluminescent engraftment analysis was performed weekly on transplanted mice with the aid of a non-invasive in vivo imaging system (IVIS 200, Caliper Inc). Mice were sacrificed 8 weeks after transplantation and human erythroid engraftment analyzed in hematopoietic organs including spleen, liver, bone marrow and thymus following staining with anti-human antibodies including CD45 and glycophorin A. FACS analysis demonstrated enhanced human erythroid (FSc^{lo}human glycophorin A⁺CD45^{-/lo}) engraftment by PV HSC and progenitors compared with their normal counterparts. D. Left panel: Representative bioluminescent images of RAG2−/−γ_{c}−/− mice transplanted with lentiviral luciferase-transduced CD34⁺ progenitor cells derived from phlebotomies donated by 4 separate JAK2 V617F+ patients before (4 weeks post-transplant) and after 12 days of treatment with vehicle or 150 mg/kg of TG101348 administered by twice daily oral gavage. Right panel: FACS analysis performed 6 weeks post-transplant, demonstrated a significant reduction (p=0.022) in the percentage of human erythroid (FSc^{lo}human glycophorin A⁺CD45^{-/lo}) cells in the livers of mice transplanted with PV progenitors and treated with TG101348 compared with vehicle treated controls.

FIGS. 4A-4B. Selective Inhibition of JAK2 V617F-Driven Erythroid Engraftment. A. Representative bioluminescent images of RAG2−/−γ_{c}−/− mice (n=4 experiments) transplanted intrahepatically with normal cord blood progenitors that were lentivirally transduced with luciferase-GFP together with a lentiviral backbone vector (BB), wild-type JAK2 (WT JAK2) or mutant JAK2 (MUT JAK2). Mice (n=24) were imaged with the aid of a non-invasive in vivo imaging system (IVIS 200, Caliper Inc) before (4 weeks post-transplant), during (Day 5 TG101348) and after (Day 12 TG101348) treatment with vehicle (DMSO) or 150 mg/kg of TG101348 administered twice daily by oral gavage. Non-transplanted littermates (no Tp) served as negative controls. There was a consistent decrease (p=0.08) in bioluminescent engraftment by mutant JAK2 progenitors following treatment with TG101348 in contrast to backbone (p=0.61) and wild-type JAK2 (p=0.67) progenitors. B. In 4 experiments, RAG2−/−γ_{c}−/− mice transplanted intrahepatically at birth with lentiviral backbone, wild type JAK2 or mutant JAK2 (V617F) transduced human cord blood progenitors were treated with vehicle or TG101348 (150 mg/kg) for 12 days by twice daily oral gavage. At 6 weeks post-transplant, FACS analysis was performed on anti-human CD45-APC and anti-human glycophorin A-Cy7-PE antibody stained mouse hematopoietic tissues including liver, bone marrow, thymus and spleen. In 4 experiments, JAK2 V617F enhanced human erythroid (CD45⁻GlycophorinA⁺) engraftment was inhibited with TG101348. Mutant JAK2-driven human erythroid engraftment was significantly inhibited (p=0.037) while wild-type JAK2 progenitor derived erythroid engraftment was inhibited to a lesser extent (p=0.077) as was erythroid engraftment by backbone transduced progenitors (p=0.27).

FIG. 5A-5D. JAK2 Driven Erythroid Signal Transduction Pathways are Inhibited by TG101348. A. Quantitative RT-PCR analysis of GATA-1 and PU.1 expression was preformed on FACS sorted hematopoietic stem cells (HSC; CD34⁺CD38⁻CD90⁺Lin⁻) and progenitor cells including common myeloid progenitors (CMP; CD34+CD38⁺IL-3Rα⁺CD45RA⁻Lin⁻), granulocyte-macrophage progenitors (GMP; CD34+CD38⁺IL-3Rα⁺CD45RA⁺Lin⁻) and megakaryocyte-erythroid progenitors (MEP; CD34+CD38⁺IL-3Rα⁻CD45RA⁻Lin⁻) from PV patient blood. Results were normalized to human HPRT expression. This analysis indicated that patients with stable PV have an increase in GATA-1 relative to PU.1 expression in keeping with their enhanced erythropoiesis. B. Quantitative RT-PCR analysis of GATA-1, PU.1 and FOG-1 expression was performed on 10⁴ CD34-enriched cord blood cells (n=2 experiments) transduced with backbone lentiviral vector (pLV) or a JAK2 V617F expressing lentiviral vector (Jak2 MT) following treatment with vehicle (DMSO) or a TG101348 (IN) for 7 days in myelocult media (Stem Cell Technologies Inc). Following treatment of Jak2MT cells with TG101348 (300 nM), GATA-1 transcripts (left panel, red) significantly decreased 25% (p=0.013) while PU.1 transcript levels did not change significantly (middle panel, blue). FOG-1 transcript levels increased by 30% (right panel, yellow) (p=0.07) while the ratio of GATA-1/FOG-1 transcript levels (orange) decreased significantly by 52% (p=0.05). A two-tailed t-test was performed with InStat analysis software. C. UT7/EPO cells were starved in 0.1% FBS in IMDM for 24 h. Prior to stimulation with erythropoietin (EPO), cells were incubated with a PI3-kinase inhibitor (LY294002; 10 μM), a non-selective JAK2 inhibitor (AG490; 50 μM) and a selective JAK2 inhibitor (TG101348; 300 and 600 nM). Cells were stimulated with EPO (10 U/ml) for 30 min. Equal amounts of lysates (20 μg) were run on a 4-15% SDS-PAGE gradient gel and probed with phospho-AKT, phospho-GATA-1 (S310) and phospho-STAT5. Blots were stripped and re-probed with AKT, GATA-1 and STAT5 antibodies. Lane 1) No EPO and no inhibitor; Lane 2-6) EPO (10 U/ml) for 1 h; Lane 2) No inhibitor; Lane 3) LY294002 (10 μM); Lane 4) AG490 (50 μM); Lane 5) TG101348 (300 nM); Lane 6) TG101348 (600 nM). D. Model of the mechanism of inhibition of JAK2 Driven erythroid differentiation by TG101348. These data suggest that TG101348 inhibits JAK2-mediated STAT5 as well as AKT mediated GATA-1 phosphorylation leading to a potent block in erythroid differentiation following erythropoietin (Epo) binding to the erythropoietin receptor (EpoR) or activation of signaling through JAK2 V617F.

FIG. 6. Inhibition of JAK2 V617F driven erythroid engraftment by TG101348. In 4 experiments, quantitative bioluminescent imaging (photons/s/cm²/sr×10⁵+/−S.E.M.) demonstrates a consistent decrease (p=0.08) in mutant JAK2 progenitor engraftment following 12 days of TG101348 administration, in contrast to backbone (p+0.61) progenitor transplanted mice treated in the same manner. Series 1 refers to before treatment while series 2 depicts response to 12 days of treatment.

FIGS. 7A-7C. Selective Inhibition of JAK2-driven engraftment. A. FACS analysis of bone marrow derived from 6 week old mice transplanted with backbone versus mutant JAK2 transduced cord blood progenitors showed a decrease in human CD45+ cells (gated on human CD45+, propidium iodide negative cells) following treatment with vehicle or TG101348 (140 mg/kg/dose) by twice daily oral gavage for 12 days. B. There was no significant decrease in human CD14 or 33 engraftment by JAK2 mutant (p=0.20) or backbone (p=0.19) expressing progenitors following treatment with TG101348. C. Lentiviral luciferase-transduced CD34+ progenitor cells were also transduced with backbone or mutant JAK2 lentiviral vectors followed by intrahepatic transplantation into newborn RAG2^{-/-}γc^{-/-} mice (n=4 per group). Bioluminscent engraftment analysis was performed with the aid of a non-invasive in vivo imaging system (IVIS 200, Caliper Inc.) before (4 weeks post-transplant) and after 14 days of treatment with vehicle or 140 mg/kg of TG101348 (6 weeks post-transplant) by twice daily oral gavage.

### DETAILED DESCRIPTION OF THE EMBODIMENTS

Hematopoietic stem and/or progenitor cells are genetically altered, e.g. by lentiviral transduction, introduction of plasmids or other vectors, including viral vectors such as AAV, adenovirus, and the like to overexpress genes involved in cell-fate determination. Genes of interest include, without limitation, human JAK2 wild-type sequence, human JAK2 V617F mutant sequence, etc. The genetically altered cells are then transplanted into an immunocompromised non-human host, e.g. (RAG2^{-/-}γc^{-/-} mice, SCID mice, etc.) The cells may be introduced intrahepatically at birth, or at other suitable times and routes, e.g. i.v., etc. Preferably the cells provide for a detectable marker, particularly a bioluminescent marker, e.g. luciferase, GFP and the like, and may be tracked in vivo via bioluminescent imaging (Caliper IVIS 200), by FACS analysis on hematopoietic organs including liver, spleen, marrow and thymus, and the like. Quantitative PCR may be performed on the genetically altered stem and/or progenitor cells as well as patient samples to corroborate changes in gene expression seen in response to overexpression of a specific gene with phenotype.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

The term “unit dosage form” as used herein refers to physically discrete units suitable as unitary dosages for subjects (e.g., animals, usually humans), each unit containing a predetermined quantity of agent(s) in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

The stem and/or progenitor cells may be obtained from any mammalian species, e.g. human, equine, bovine, porcine, canine, feline, rodent, e.g. mice, rats, hamster, primate, etc., usually human. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−180° C.) indefinitely. The population of stem and/or progenitor cells may be enriched for a population of interest, although such an enrichment step is not required, particularly where the tissue provides for significant numbers of stem cells, e.g. bone marrow, cord blood, fetal liver, and the like. A tissue of particular interest is human cord blood.

Hematopoietic stem cells (HSCs) can be functionally defined by their unique capacity to self-renew, and to differentiate to produce all mature blood cell types. In general, the process of development from pluripotent progenitors to mature cells with specific functions involves the progressive loss of developmental potential to other lineages. A hierarchy has emerged in which each successive developmental stage loses the potential to become a specific cell type or class of cells. This stepwise developmental process has been considered linear in the sense that once a cell has made a developmental choice it cannot revert. The earliest known lymphoid-restricted cell in adult mouse bone marrow is the common lymphocyte progenitor (CLP), and the earliest known myeloid-restricted cell is the common myeloid progenitor (CMP). Importantly, these cell populations possess an extremely high level of lineage fidelity in in vitro and in vivo developmental assays. A complete description of these cell subsets may be found in Akashi et al. (2000) Nature 404(6774):193, U.S. Pat. No. 6,465,247; published patent application U.S. Ser. No. 09/956,279 (common myeloid progenitor); Kondo et al. (1997) Cell 91(5):661-7, and International application WO99/10478 (common lymphoid progenitor); and is reviewed by Kondo et al. (2003) Annu Rev Immunol. 21:759-806, each of which is herein specifically incorporated by reference.

CD34+ hematopoietic cells harbor virtually all in vitro hematopoietic clonogenic potential; however, the CD34+ population is heterogeneous. Only a small fraction (1-10%) of CD34+ cells that do not express mature lineage markers (Lin⁻, including the markers CD3, CD4, CD8, CD19, CD20, CD56, CD11b, CD14, and CD15) have multilineage (lymphoid and myeloid) developmental potential. The majority of CD34+ cells (90-99%) coexpress the CD38 antigen, and this subset contains most of the lineage-restricted progenitors.

In the myeloid lineage are three cell populations, termed CMPs, GMPs, and MEPs. These cells are CD34⁺ CD38⁺, they are negative for multiple mature lineage markers including early lymphoid markers such as CD7, CD10, and IL-7R, and they are further distinguished by the markers CD45RA, an isoform of CD45 that can negatively regulate at least some classes of cytokine receptor signaling, and IL-3R. These characteristics are CD45RA⁻ IL-3Rα^{lo} (CMPs), CD45RA⁺IL-3Rα^{lo} (GMPs), and CD45RA⁻IL-3Rα⁻ (MEPs). CD45RA⁻ IL-3Rα^{lo} cells give rise to GMPs and MEPs and at least one third generate both GM and MegE colonies on a single-cell level.

In both human and mouse cells, all three of the myeloid lineage progenitors stain negatively for the markers Thy-1 (CD90), IL-7Rα (CD127); and with a panel of lineage markers, which lineage markers may include CD2; CD3; CD4; CD7; CD8; CD10; CD11b; CD14; CD19; CD20; CD56; and glycophorin A (GPA) in humans. With the exception of the mouse MEP subset, all of the progenitor cells are CD34 positive. In the human all of the progenitor subsets are CD38⁺.

Common lymphoid progenitors, CLP, express low levels of c-kit (CD117) on their cell surface. Antibodies that specifically bind c-kit in humans, mice, rats, etc. are known in the art. Alternatively, the c-kit ligand, steel factor (SO may be used to identify cells expressing c-kit. The CLP cells express high levels of the IL-7 receptor alpha chain (CDw127). Antibodies that bind to human or to mouse CDw127 are known in the art. Alternatively, the cells are identified by binding of the ligand to the receptor, IL-7. Human CLPs express low levels of CD34. Antibodies specific for human CD34 are commercially available and well known in the art. See, for example, Chen et al. (1997) Immunol Rev 157:41-51. Human CLP cells are also characterized as CD38 positive and CD10 positive.

The CLP subset also has the phenotype of lacking expression of lineage specific markers, exemplified by B220, CD4, CD8, CD3, Gr-1 and Mac-1. The CLP cells are characterized as lacking expression of Thy-1, a marker that is characteristic of hematopoietic stem cells. The phenotype of the CLP may be further characterized as MeI-14⁻, CD43^{lo}, HSA^{lo}, CD45⁺ and common cytokine receptor γ chain positive.

The analysis of megakaryocyte progenitors may also be of interest. The MKP cells are positive for CD34 expression, and tetraspanin CD9 antigen. The CD9 antigen is a 227-amino acid molecule with 4 hydrophobic domains and 1 N-glycosylation site. The antigen is widely expressed, but is not present on certain progenitor cells in the hematopoietic lineages. The MKP cells express CD41, also referred to as the glycoprotein IIb/IIIa integrin, which is the platelet receptor for fibrinogen and several other extracellular matrix molecules, for which antibodies are commercially available, for example from BD Biosciences, Pharmingen, San Diego, Calif., catalog number 340929, 555466. The MKP cells are positive for expression of CD117, which recognizes the receptor tyrosine kinase c-Kit. Antibodies are commercially available, for example from BD Biosciences, Pharmingen, San Diego, Calif., Cat. No. 340529. MKP cells are also lineage negative, and negative for expression of Thy-1 (CD90).

A suitable population of hematopoietic cells, includes, without limitation, human cord blood, mobilized human peripheral blood, human bone marrow, human fetal liver, and the like, any of which may be used without specific enrichment for hematopoietic stem or progenitor cells. Alternatively the cell population is selectively enriched for a stem or progenitor cell of interest, e.g. HSC, CMP, CLP, MEP, etc., using any combination of markers as described above for selective enrichment, e.g. using magnetic sorting techniques, flow cytometry, etc. as known in the art. In some embodiments the cells are enriched for expression of CD34, e.g. by immunomagnetic selection, flow cytometry, etc. Selection may be performed before or after introduction and expression of a JAK2 encoding nucleic acid construct.

Separation of the desired cells for engraftment is optional, may be performed using affinity separation to provide an enriched cell opulation with a phenotype of interest, as described above for hematopoietic stem and/or progenitor cells. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, LDS). Any technique may be employed which is not unduly detrimental to the viability of the selected cells.

Of particular interest is the use of antibodies as affinity reagents. Conveniently, these antibodies are conjugated with a label for use in separation or used in conjunction with a labeled second antibody that binds to them. Labels include magnetic beads, which allow for direct separation; biotin, which can be bound to avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red.

A nucleic acid construct is introduced into the above-described cell or population of cells, where the construct comprises sequences that overexpress genes involved in cell-fate determination. Genes of interest include, without limitation, human JAK2 wild-type sequence, human JAK2 V617F mutant sequence. The human JAK2 V617F mutant sequence is of particular interest. The sequence of human JAK2 is known and publicly available, for example at Genbank accession number AY973034, and the V617F mutation is described by Baxter et al. (2005) Lancet 365, 1054-1061 and Kralovics et al. (2005) New Eng. J. Med. 352: 1779-1790. The coding sequence is operably linked to a promoter, which may be a constitutive or inducible promoter, and may be the native promoter for the gene of interest, or may be heterologous relative to the coding sequence. A construct of interest contains human JAK2 V617F and its native promoter.

A variety of vectors are known in the art for the delivery of sequences into a cell, including plasmid vectors, viral vectors, and the like. In a preferred embodiment, the vector is a retroviral or lentiviral vector. For example, see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210, Mochizuki et al. (1998) J Virol 72(11):8873-83. The use of adenovirus based vectors with hematopoietic cells has also been published, see Ogniben and Haas (1998) Recent Results Cancer Res 144:86-92.

Combinations of retroviruses and an appropriate packaging line may be used, where the capsid proteins will be functional for infecting the target cells. Usually, the cells and virus will be incubated for at least about 24 hours in the culture medium. Commonly used retroviral vectors are “defective”, i.e. unable to produce viral proteins required for productive infection. Replication of the vector requires growth in the packaging cell line.

Various techniques known in the art may be used to transfect the target cells, e.g. electroporation, calcium precipitated DNA, fusion, transfection, lipofection and the like. The particular manner in which the DNA is introduced is not critical to the practice of the invention.

The nucleic acid construct may further comprise a detectable marker. Viable cells expressing the marker can also be sorted, in order to isolate or enrich for the cells of interest. Many such markers are known in the art, for example antibiotic resistance, color change of a substrate, expression of a recombinase, e.g. cre recombinase, FLP recombinase, pSR1 recombinase, etc., which is indirectly detected; expression of luminescence producing proteins, e.g. luciferase, green fluorescent proteins, etc.

In a preferred embodiment of the invention, the marker is a luminescence producing protein, preferably green fluorescent protein (GFP) and/or luciferase. Luciferase, e.g. firefly luciferase enzyme, operably linked to a constitutive or inducible promoter allows in vivo bioluminescence detection of transfected cells, for example using a CCCD camera after luciferin administration. See Contag et al. (1998) Nat. Med. 4:245, herein specifically incorporated by reference for teaching the use of luciferase transgenes.

The native gene encoding GFP has been cloned from the bioluminescent jellyfish Aequorea victoria (Morin, J. et al., J Cell Physiol (1972) 77:313-318). The availability of the gene has made it possible to use GFP as a marker for gene expression. GFP itself is a 283 amino acid protein with a molecular weight of 27 kD. It requires no additional proteins from its native source nor does it require substrates or cofactors available only in its native source in order to fluoresce. Mutants of the GFP gene have been found useful to enhance expression and to modify excitation and fluorescence. GFP-S65T (wherein serine at 65 is replaced with threonine) may be used, which has a single excitation peak at 490 nm. (Heim, R. et al., Nature (1995) 373:663-664); U.S. Pat. No. 5,625,048. Other mutants have also been disclosed by Delagrade, S. et al., Biotechnology (1995) 13:151-154; Cormack, B. et al., Gene (1996) 173:33-38 and Cramer, A. et al. Nature Biotechnol (1996) 14:315-319. Additional mutants are also disclosed in U.S. Pat. No. 5,625,048. By suitable modification, the spectrum of light emitted by the GFP can be altered.

The expression of the detectable marker, where the marker is a fluorescent protein, can be monitored by CCCD camera, flow cytometry, where lasers detect the quantitative levels of fluorophore. Flow cytometry, or FACS, can also be used to separate cell populations based on the intensity of fluorescence, as well as other parameters such as cell size and light scatter. Although the absolute level of staining may differ, the data can be normalized to a control.

The genetically modified cells are introduced into a suitable xenogeneic immunocompromised animal host. Any suitable site for injection may be used, e.g. intravenous, or to a hematopoietic organ, e.g. intrathymic, intra-marrow, intrasplenic, intrahepatic, etc. In some embodiments intrahepatic injection is preferred. Various numbers of cells are introduced, depending on the specific use of the animal model, and the purity of cells being transplanted, where highly purified hematopoietic stem cells may be dministered in lower numbers than unselected populations. Usually at least about 10, 10², 10³, 10⁴, 10⁵ 10⁶ or more cells are introduced.

Immunocompromised mammalian hosts suitable for implantation and having the desired immune incapacity exist or can be created. The significant factor is that the immunocompromised host is incapable of mounting an immune response against the introduced cells. Of particular interest are small mammals, e.g. rabbits, gerbils, hamsters, guinea pigs, etc., particularly rodents, e.g. mouse and rat, which are immunocompromised due to a genetic defect that results in an inability to undergo germline DNA rearrangement at the loci encoding immunoglobulins and T-cell antigen receptors or to a genetic defect in thymus development (nu/nu). Mazurier (1999) J Interferon Cytokine Res 19: 533-41 developed an immunodeficient mouse model by combining recombinase activating gene-2 (RAG2) and common cytokine receptor gamma chain (gamma c) mutations. The RAG2^{-/-}γc^{-/-} double mutant mice are completely alymphoid (T-, B-, NK-), show no spontaneous tumor formation, and exhibit normal hematopoietic parameters, and are of particular interest.

Presently available hosts also include mice that have been genetically engineered by transgenic disruption to lack the recombinase function associated with RAG-1 and/or RAG-2 (e.g. commercially available TIM™ RAG-2 transgenic), to lack Class I and/or Class II MHC antigens (e.g. the commercially available C1D and C2D transgenic strains), or to lack expression of the Bcl-2 proto-oncogene. In other embodiments the host animals are mice that have a homozygous mutation at the scid locus, causing a severe combined immunodeficiency which is manifested by a lack of functionally recombined immunoglobulin and T-cell receptor genes. The scid/scid mutation is available or may be bred into a number of different genetic backgrounds, e.g. CB.17, ICR (outbred), C3H, BALB/c, C57Bl/6, AKR, BA, B10, 129, etc. Other mice which are useful as recipients are NOD scid/scid; SGB scid/scid, bh/bh; CB.17 scid/hr; NIH-3 bg/nu/xid and META nu/nu. Transgenic mice, rats and pigs are available which lack functional B cells and T cells due to a homozygous disruption in the CD3ε gene. Immunocompromised rats include HsdHan:RNU-rnu; HsdHan:RNU-rnu/+; HsdHan:NZNU-mu; HsdHan:NZNU-rnu/+; LEW/HanHsd-rnu; LEW/HanHsd-rnu/+; WAG/HanHsd-rnu and WAG/HanHsd-rnu/+.

The host may be neonate, or newborn to young adult, usually less than about 6 weeks of age, and may be less than about 4 weeks of age, less than about 2 weeks of age at implantation. The mammalian host will be grown in conventional ways. Depending on the degree of immunocompromised status of the mammalian host, it may be protected to varying degrees from infection. An aseptic environment is indicated. Prophylactic antibiosis may be used for protection from infection. Alternatively, it may be satisfactory to isolate the potential hosts from other animals in gnotobiotic environments after cesarean derivation. The feeding and maintenance of the host will for the most part follow gnotobiotic techniques.

The subject JAK2 overexpressing cells and animal models are useful for in vitro and in vivo assays and screening to detect factors that are active in influencing the erythroid pathway. The cells find use in assessing pre-clinical efficacy of anti-JAK2 drugs alone and in combination with other agents. The cells provide a model for testing anti-malarial drugs and other pathogens that affect developing red blood cells. The cells provide a model for assessing drug toxicity toward myeloid and erythroid progenitors. The model may be used to develop novel diagnostics to detect aberrant JAK2 signaling in hematopoietic stem and progenitor cells. In addition, the models provides for assessment of agonists of hematopoietic stem cell as well as myeloid and erythroid progenitor differentiation such as hematopoietic growth factors.

In screening assays of particular interest, an inhibitor of JAK2 function is administered to the animal, which may be administered at a dose effective to substantially inhibit JAK2 activity, or may be administered at various concentrations. In some embodiments, a known JAK2 inhibitor, e.g. TG101348, which provides a useful model for treatment of myeloproliferative disorders associated with JAK2, and which also provides a control for the study of such myeloproliferative disorders where, for example, a panel of animals may be used in screening of candidate agents, where the animals in the panel comprise a mutant JAK2 mutation, and where at least one of said animals has been treated with a known JAK2 inhibitor.

In some embodiments, the animals or a panel of animals are contacted with a candidate agent for the treatment of myeloproliferative JAK2 associated disease, where the candidate agent is administered to the animal in a suitable manner, e.g. orally, by injection, inhalation, etc., and the effect on erythropoiesis is examined following such administration, by determining the quantity and/or characteristics of progeny from the JAK2 expressing xenogeneic cells.

The animal models of the invention may be used to test novel therapeutics directed against JAK2 signaling. Of particular interest are screening assays for agents that are active on human cells. A wide variety of assays may be used for this purpose, including immunoassays for protein binding; determination of cell growth, differentiation and functional activity; production of cytokines, e.g. IL-1; expression and phosphorylation of transcriptional factors involved in lineage determination, and the like.

Parameters of interest for assessment of a candidate agent include the quantitation of xenogeneic erythrocytes, which may be quantitated by the number of glycophorin expressing cells, e.g. staining with antibodies specific for human glycophorin A. Such staining may further utilize the quantitation of total xenogeneic hematopoietic cells, e.g. using a detectable marker provided in the nucleic acid construct, as described above, or with other suitable markers of the xenogeneic cells, e.g. CD45, etc. Various hematopoietic organs may be collectively or individually assessed in this manner, e.g. blood, spleen, liver, bone marrow, thymus, lymph nodes, and the like.

Parameters of interest also include the expression of transcription factors involved in hematopoietic determination, erythroid (GATA-1), myeloid (PU.1). megakaryocyte (FOG-1), etc. in the xenogeneic cells. Expression may be detected by quantitative PCR, in situ hybridization, and the like, as known in the art. The cells to be analyzed are optionally selected from the hematopoietic organs. Such transcriptional factors may also be assessed for phosphorylation at the protein level.

These animals provide a useful model for polycythemia, and for erythroid disease, including infection. By providing a accurate model for the human disease, potential therapeutics can be evaluated in the animal model for safety and efficacy prior to clinical trials. In addition to screening candidate pharmaceutical agents, the subject animals are useful in determining the role of “triggering” agents in development of disease, and in the evaluation of pathogens.

The present invention also provides a non-human animal model of malaria, e.g., Plasmodium, particularly Plasmodium falciparum and is useful for identifying candidate therapeutic agents, e.g., agents having anti-pathogenic activity against Plasmodium. The human erythrocytes generated in the non-human mammal comprising JAK2 transfected hematopoietic stem or progenitor cells provide a model for infection with plasmodium, which can be infected via the normal route of infection, e.g., intravenous delivery of malarial sporozoites from Anopheline mosquitoes; or with erythrocytic stage P. falciparum in vitro and then injected into the subject animals to establish infection. In some embodiments, the animals further comprise human liver tissue transplanted by methods known in the art, which allow the complete malarial life cycle to be recapitulated in an animal model.

“Malarial parasite” as used herein, and unless specifically indicated otherwise, and “human malarial parasite” as used herein generally refer to a parasite species of the genus Plasmodium which is a causative agent of protozoan disease in humans. There are at least four species of Plasmodium which are currently known to cause malaria in humans: P. falciparum; P. vivax; P. ovale; and P. malariae. The parasites can be naturally transmitted to the human host by the bite of an infected female mosquito of the genus Anopheles. “Human malarial parasite” is not intended to limit the parasites to those immediately recovered for humans; rather it is intended to refer to malarial parasites that can cause human disease. Such human malarial parasites are not normally infectious for non-primate animals.

The term “infection” as used in the context of an animal model described herein infected with a malarial parasite, is meant to refer to the state of an animal from which malarial parasites can be recovered, where the animal may or may not exhibit any or all clinical symptoms associated with malarial infection.

Methods for obtaining malarial parasites and administration to a mammal are well known in the art. For example, infected Anopheline mosquitoes are obtained from the Malaria Program at the Naval Medical Research Center, Silver Spring, Md. The mosquitoes are microdissected and the salivary glands, containing the sporozoites are triturated in a ground-glass homgenizer, counted and injected intravenously (0.1−3×10⁶ parasites/mouse) into the mice. The number of parasites that are injected depends upon the specific study as well as the recovery of sporozoites from the mosquitoes. The specific procedures for the recovery of P. falciparum sporozoites are well known in the art and are common practice for investigators in the field of Plasmodium.

P. falciparum parasite takes approximately 7-9 days to mature in the hepatocyte and will subsequently be released from the hepatocyte to infect erythrocytes. Thus, there is no chronic state of infection with P. falciparum. However P. vivax can form hypnozoites that remain in the liver in a developmentally arrested state for an extended period of time (months to years). For reasons that are not understood, P. vivax hypnozoites will re-initiate development and mature in the hepatocyte. The ability to study this phenomenon an advantage of the invention, as such can not be studied in an in vitro system but can be assessed in the chimeric animal model of the invention.

The parasitic load of the infected host can be determined. The parasitic load can be determined either qualitatively or quantitatively by, for example, examination of tissue (e.g., liver cells, blood smears), PCR (e.g., real-time PCR assays), and the like. For example, real-time quantitative PCR (RTQ-PCR) can be utilized to determine the parasite burden in the livers and blood of P. falciparum infected chimeric animals. The small subunit (18S) ribosomal RNA gene of Plasmodium is a well conserved gene that can be used as a target for RTQ-PCR. For quantitative analysis, a standard curve can be constructed using DNA that is extracted from known concentrations of blood stage parasites. DNA from liver stage samples is extracted and used in a RTQ-PCR assay with DNA from known concentrations of parasites. A cycle threshold (Ct) versus parasite number is produced for the standard and the number of parasites in the liver calculated by plotting the CT of the liver stage samples.

The parasitic load of the infected host over time can mimic that observed in human infection, which correlates with the development of the parasite during is natural life cycle. Thus the invention encompasses chimeric hosts having EE stage parasites (liver stage parasites, including developing intrahepatic merozoites) as well as erythrocytic stage (or “red cell” stage) parasites (including trophozoites). In general, the chimeric animal can be provided so as to support all or part (e.g., pre-erythrocytic) of the malarial life cycle. That is, the animal model of the invention can support the entire life cycle of the malarial parasite (e.g., P. falciparum) through all stages, which can include transfer of blood-borne parasites from one infected chimeric animal to another (e.g., by artificial transfer for by mosquito).

Infection of the chimeric animals of the invention can be established with introduction into the animal (e.g., into the animal's bloodstream) of at least about 10, 100, 5×10², 10³, 5×10³, 10⁴, 5×10⁴, 10⁵, 5×10⁵, 10⁶, 5×10⁶ parasites. In some embodiments, malarial infection of a chimeric animal of the invention is maintained for at least 3 days, 5 days, 6 days, 7 days, 8 days or more, where longer periods of infection which involve the erythrocytic stage of the parasite are maintained.

The animals of the invention can be used in a variety of screening assays suitable for identification of agents that inhibit malarial infection, development, replication, and the like. To this end, the animal model of the invention is used to screen candidate agents for such effects.

The screening assays described herein can be used with chimeric animals having a malarial infection at any stage of the malarial life cycle, including the EE stage of infection (which includes the liver stage) and the erthrocytic stage of infection. Thus the invention encompasses screening for agents that affect a malarial parasite at any stage, including to inhibit infection of hepatocytes by sporozoites, inhibit development within hepatocytes, inhibit release of merozoites from hepatocytes, inhibit infection of red blood cells by merozoites, inhibit development within red blood cells, inhibit release of merozoites, and the like.

The candidate agent can be administered in any manner desired and/or appropriate for delivery of the agent in order to effect a desired result. For example, the candidate agent can be administered by injection (e.g., by injection intravenously, intramuscularly, subcutaneously, or directly into the tissue in which the desired affect is to be achieved), orally, or by any other desirable means. Normally, the in vivo screen will involve a number of animals receiving varying amounts and concentrations of the candidate agent (from no agent to an amount of agent that approaches an upper limit of the amount that can be delivered successfully to the animal), and may include delivery of the agent in different formulations and routes. Moreover, the agents may be administered to the animals at various stages of the parasite's life cycle, as described above, with administration when the host contains liver stage parasites being of particular interest. The agents can be administered singly or can be combined in combinations of two or more, especially where administration of a combination of agents may result in a synergistic effect.

The activity of the candidate agent can be assessed in a variety of ways. For example, the effect of the agent can be assessed by examining blood samples for the presence of the parasite (e.g., titer) or markers associated with the presence of the pathogen (e.g., a pathogen-specific protein (P. falciparum histidine-rich protein II) or encoding nucleic acid, etc.) Qualitative and quantitative methods for detecting and assessing the presence and severity of malarial infection are well known in the art. The best known technology for the rapid determination of plasmodial infection is the immunochromatographic strip. In this format a monoclonal antibody that is specific for a P. falciparum protein is immobilized onto a nitrocellulose strip and is used to capture an antigen that is found in the blood of an infected individual. The prevailing test uses capture of Pf histidine-rich protein. In field studies, the test strips have been shown to be capable of detecting <500 parasites/μl. This is an exemplary technology that can be utilized to assess parasite levels in the blood of infected chimeric animals of the invention.

Drug screening protocols for malaria or myeloproliferative disease will generally include one or a panel of animals, for example a test compound or combination of test compounds, and negative and/or positive controls, where the positive controls may be known agents. Such panels may be treated in parallel, or the results of a screening assay may be compared to a reference database.

A wide variety of assays may be used for this purpose, including histological analysis of effectiveness, determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cells derived therefrom, particularly hematopoietic cells, e.g. erythrocytes, megakaryocytic cells, etc. Cells may be freshly isolated from an animal, or may be immortalized in culture. Candidate therapies may be novel, or modifications of existing treatment options.

For screening assays that use whole animals, a candidate agent or treatment is applied to the subject animals. Typically, a group of animals is used as a negative, untreated or placebo-treated control, and a test group is treated with the candidate therapy. Generally a plurality of assays are run in parallel with different agent dose levels to obtain a differential response to the various dosages. The dosages and routes of administration are determined by the specific compound or treatment to be tested, and will depend on the specific formulation, stability of the candidate agent, response of the animal, etc.

The analysis may be directed towards determining effectiveness in prevention of disease induction, where the treatment is administered before induction of the disease. Alternatively, the analysis is directed toward regression of existing disease, and the treatment is administered after initial onset of the disease, or establishment of moderate to severe disease. Frequently, treatment effective for prevention is also effective in regressing the disease.

In either case, after a period of time sufficient for the development or regression of the disease, the animals are assessed for impact of the treatment, by visual, histological, immunohistological, and other assays suitable for determining effectiveness of the treatment. The results may be expressed on a semi-quantitative or quantitative scale in order to provide a basis for statistical analysis of the results.

The term “agent” as used herein describes any molecule, e.g. protein or pharmaceutical, with the capability of affecting the severity of chronic inflammatory disease. An agent or treatment, e.g. UV light, is administered to an animal of the invention, or to cells derived therefrom. Antibodies specific for cytokines, polyclonal activating agents, and T cell antigens are agents of particular interest. Most preferably, according to another aspect of the instant invention, the agents are monoclonal antibodies, e.g. which neutralize lymphokines or block adhesion molecules.

Other candidate agents encompass numerous chemical classes, typically organic molecules. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The therapeutic agents may be administered to patients in a variety of ways, orally, topically, parenterally e.g. subcutaneously, intramuscularly, intravascularly, etc. Depending upon the manner of introduction, the compounds may be formulated in a variety of ways. The concentration of therapeutically active agent in the formulated pharmaceutical compositions may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### Animal Model for Erythroid Disease

### Results

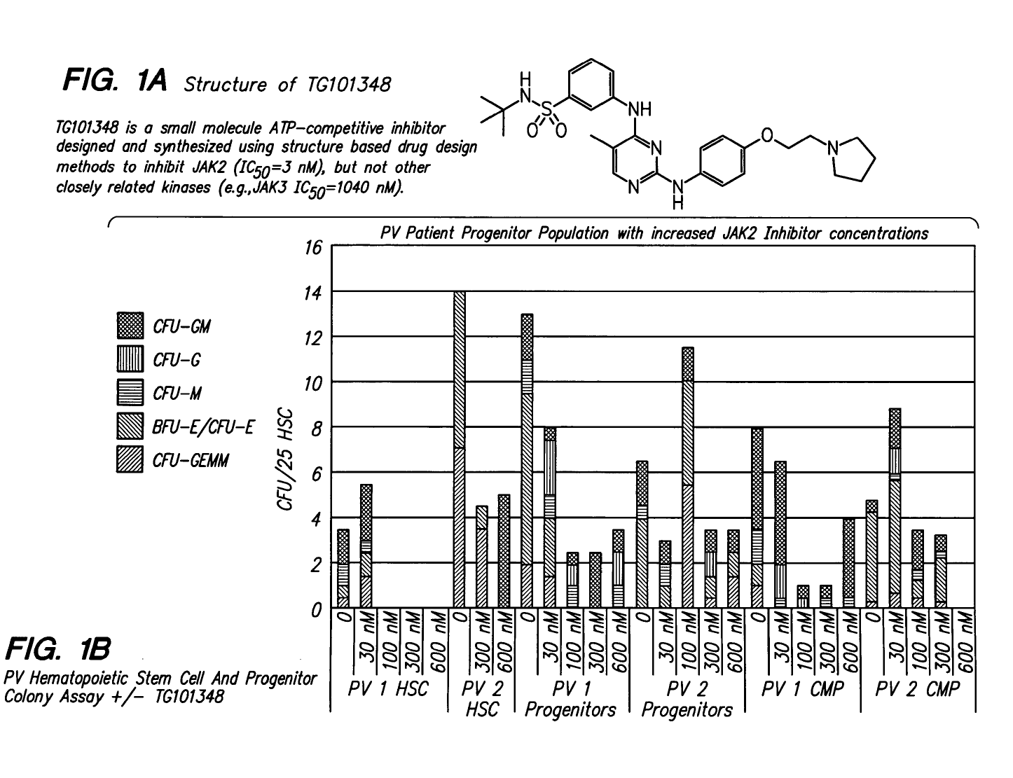

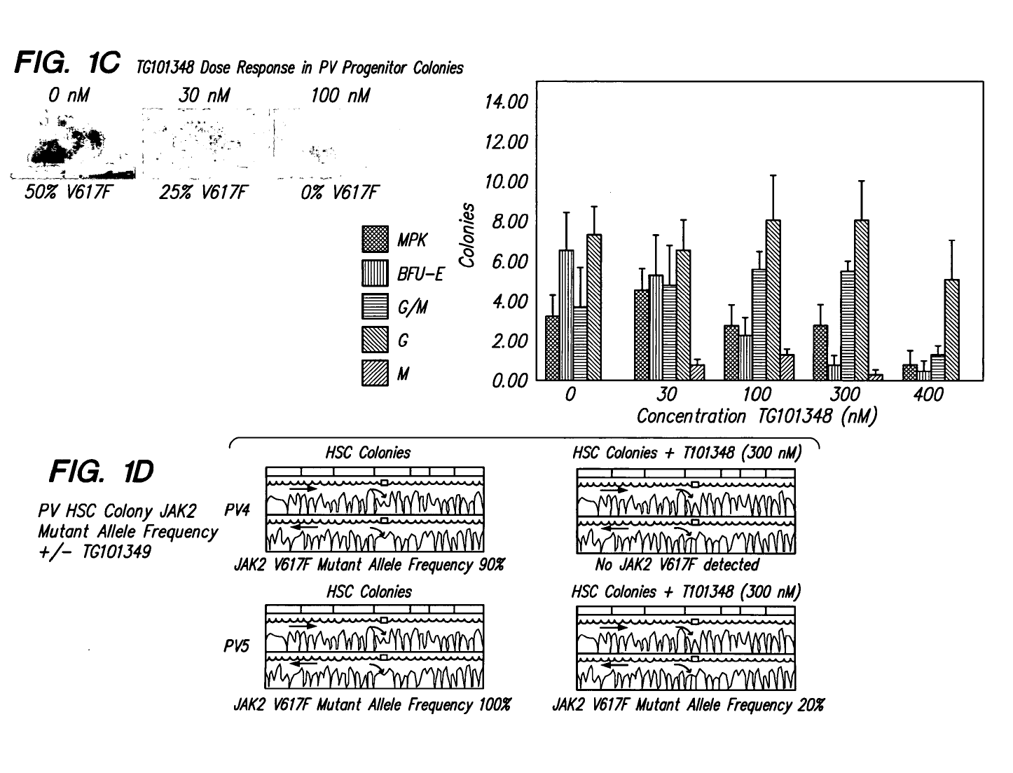

FACS analysis of early phase PV patient samples revealed an expansion in the number of cells with a HSC phenotype (CD34⁺CD38⁻CD90⁺Lin⁻) suggesting that HSC harbored an intrinsic proliferative defect. In hematopoietic progenitor assays, PV HSC gave rise to a preponderance of large erythroid colonies that expressed the JAK2 V617F mutation. In this study, we examined the erythroid differentiation potential of JAK2 V617F⁺ PV stem and progenitor cells in the presence of varying concentrations of a selective, orally available JAK2 inhibitor (TG101348; TargeGen Inc). TG101348 was designed and synthesized at TargeGen using structure based drug design methods to inhibit JAK2 and JAK2 V617F kinase (IC₅₀=3 nM for both). In contrast to other currently available inhibitors, TG101348 does not potently inhibit other closely related kinases such as JAK3 (IC₅₀=1040 nM). In four experiments, HSC(CD34⁺CD38⁻CD90⁺Lin⁻), progenitors (CD34⁺CD38⁺Lin⁻) or common myeloid progenitor (CMP; CD34⁺CD38⁺CD123⁺CD45RA⁻Lin⁻) cells from JAK V617F⁺ PV patients were clone-sorted with the aid of a FACS Aria and treated with 0, 30, 100, 300 or 600 nM of TG101348 in human cytokine supplemented methylcellulose and differential colony counts were performed on day 14. These experiments demonstrated that erythroid-skewed differentiation potential of PV progenitors was inhibited by TG101348 at a dose that was also capable of eliminating JAK2 V617F⁺ colonies although individual variability in sensitivity to TG101348 was detected (FIGS. 1A, B and C).

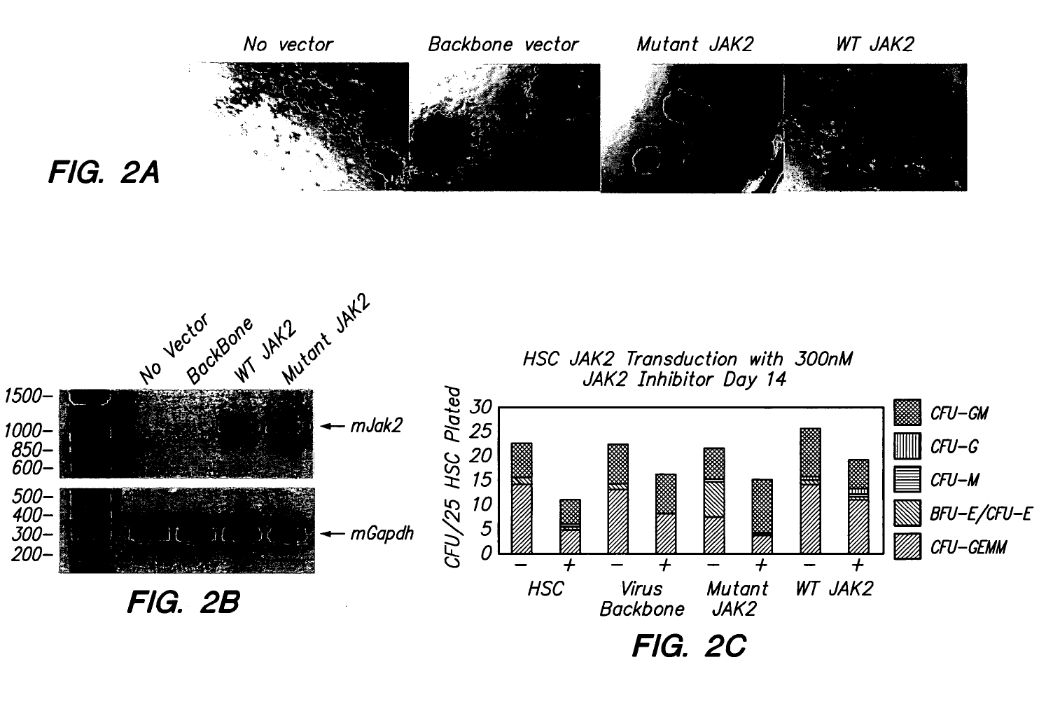

In Vitro Inhibition of JAK2 V617F-Driven Erythroid Differentiation with TG101348. The direct contribution of JAK2 V617F (mutant) to erythroid-skewed differentiation was investigated by lentivirally-enforced expression of mutant versus wild type JAK2 in normal cord blood progenitors in hematopoietic progenitor assays. Mutant JAK2 expressing cord blood progenitors gave rise to a preponderance of erythroid (BFU-E) colonies while wild-type JAK2 induced more mixed (CFU-GEMM) colony formation than backbone vector controls (n=4 experiments). PCR with primers specific for lentivirally introduced JAK2 (mJAK2) verified transduction of colonies with the lentiviral vectors. In subsequent in vitro experiments (n=4), human cord blood HSC(CD34⁺CD38⁻CD90⁺Lin⁻) transduced with lentiviral backbone, JAK2 V617F (mutant JAK2) or wild-type JAK2 (WT JAK2) vectors were treated with or without 300 nM of TG101348 (FIGS. 2A, B and C). These experiments demonstrated selective inhibition of mutant JAK2 skewed erythroid colony formation with TG101348.

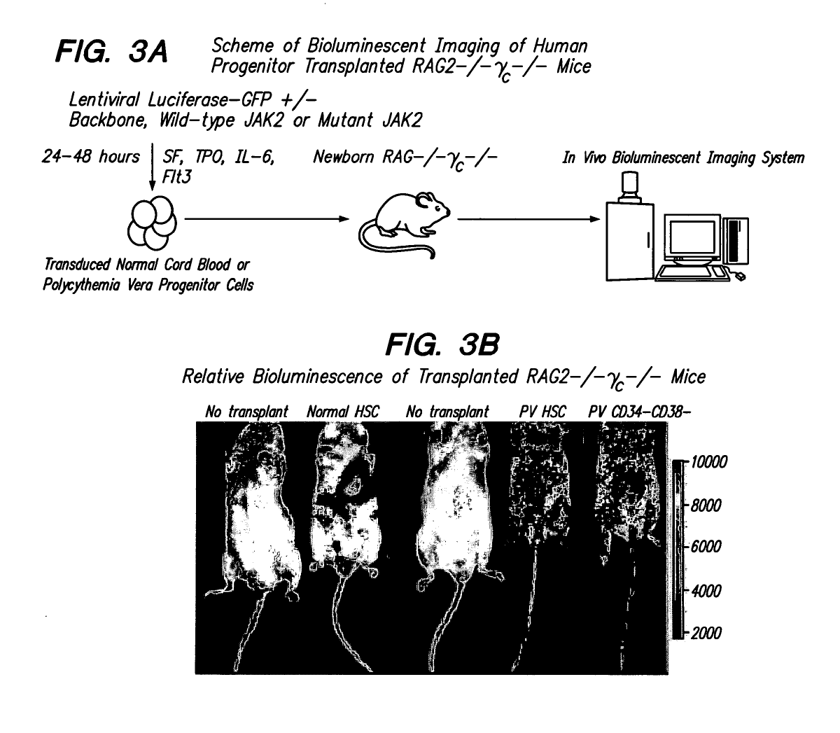

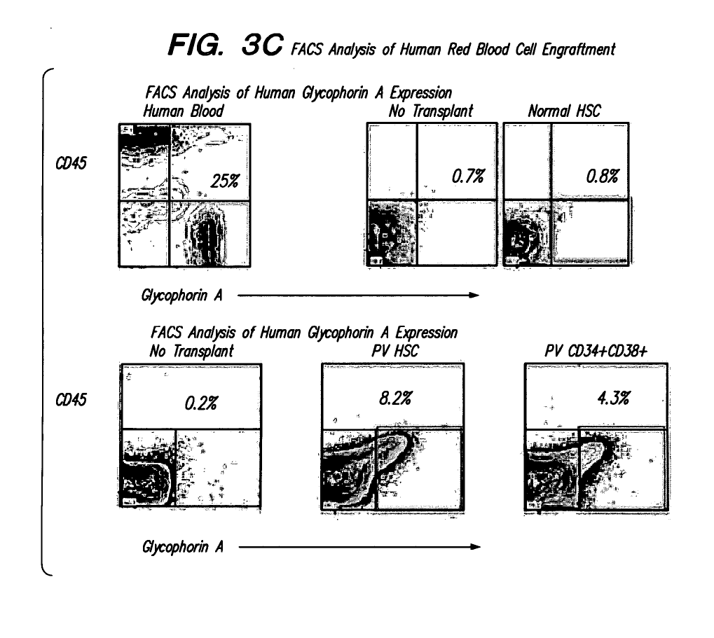

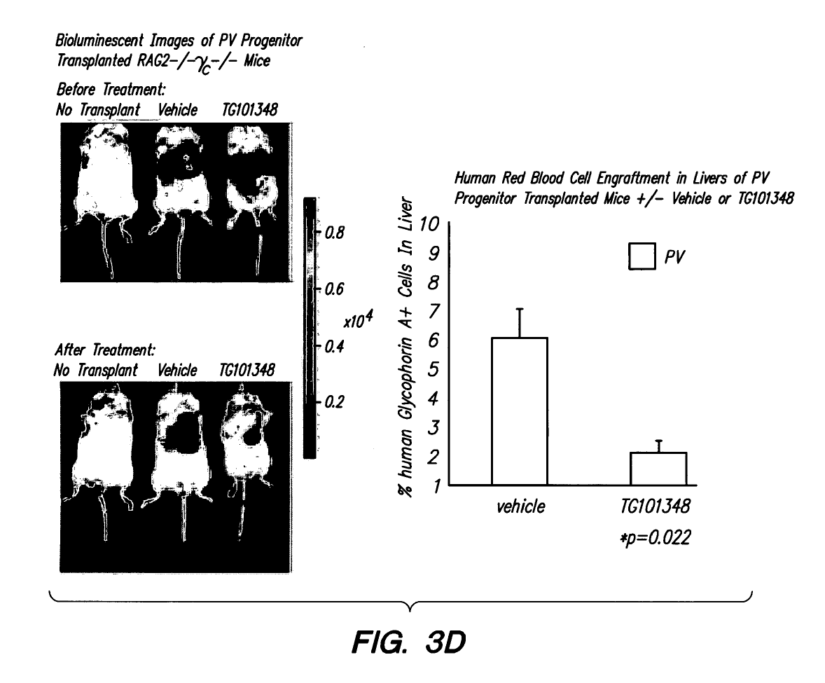

Inhibition of Human PV Progenitor Erythroid Engraftment by TG101348. The capacity of PV stem and progenitor cells to give rise to human erythroid engraftment compared with their normal counterparts was assessed in a novel bioluminescent xenogeneic transplantation model involving intrahepatic transplantation of neonatal highly immunocompromised (RAG2^{-/-}γ_{c}^{-/-}) mice with luciferase-transduced human progenitor cells (FIG. 3A). While bioluminescent imaging demonstrated comparable rates of engraftment between normal and PV progenitors (FIG. 3B), FACS analysis of engrafted hematopoietic organs revealed a skew toward in vivo erythroid differentiation by PV progenitors in hematopoietic organs of transplanted mice (FIG. 3C). In four separate experiments, oral gavage administration of TG101348 (150 mg/kg) significantly inhibited PV progenitor erythroid differentiation in vivo (FIG. 3D).

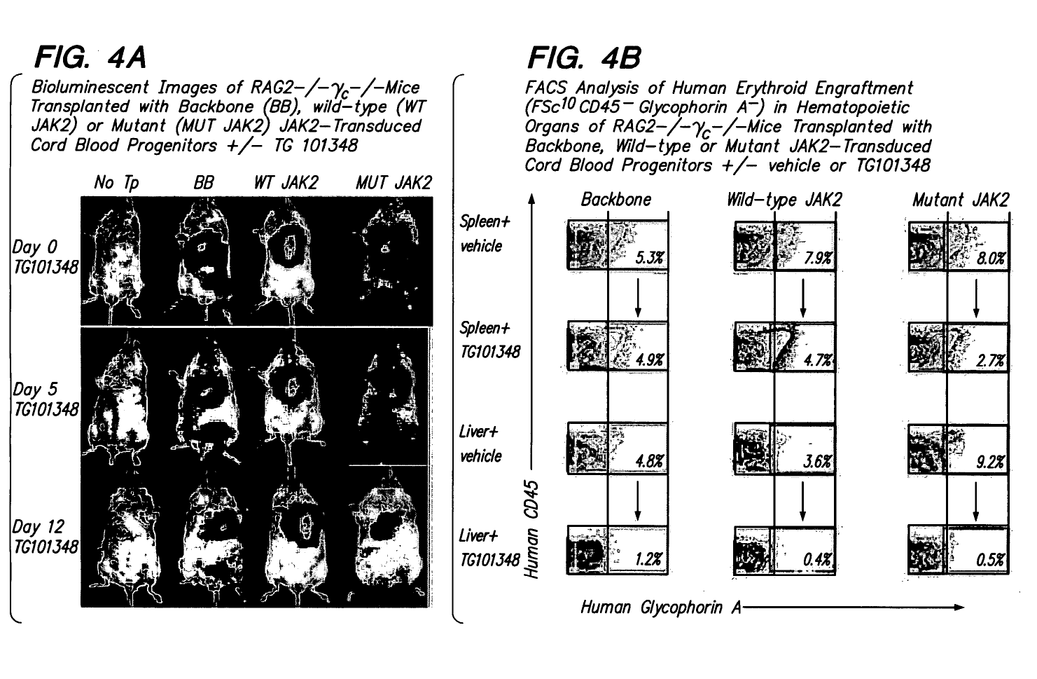

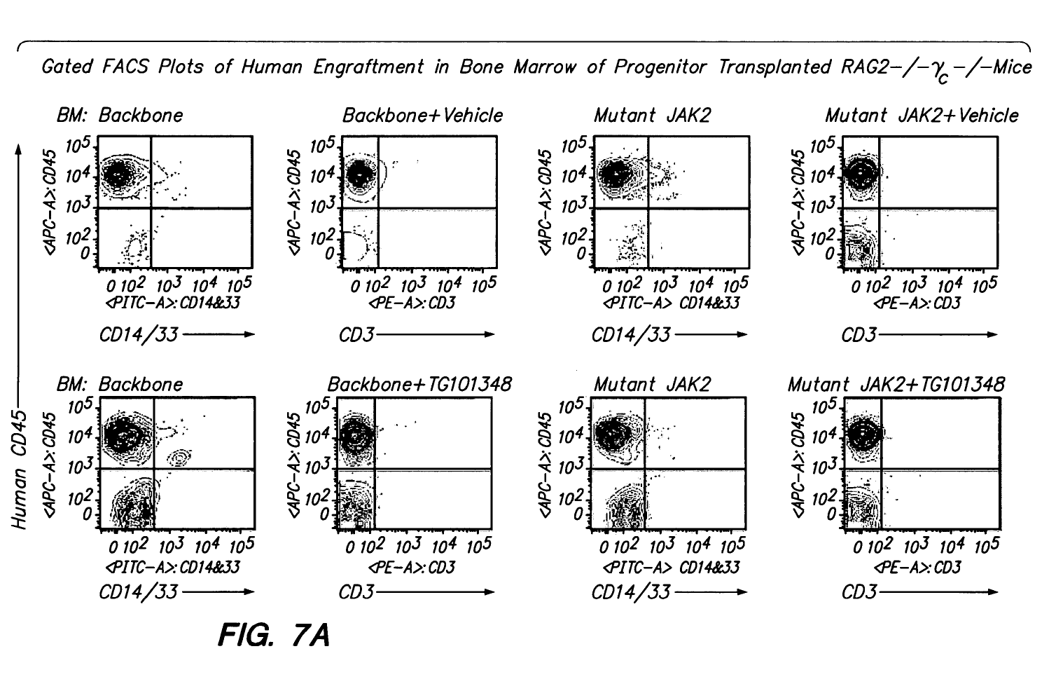

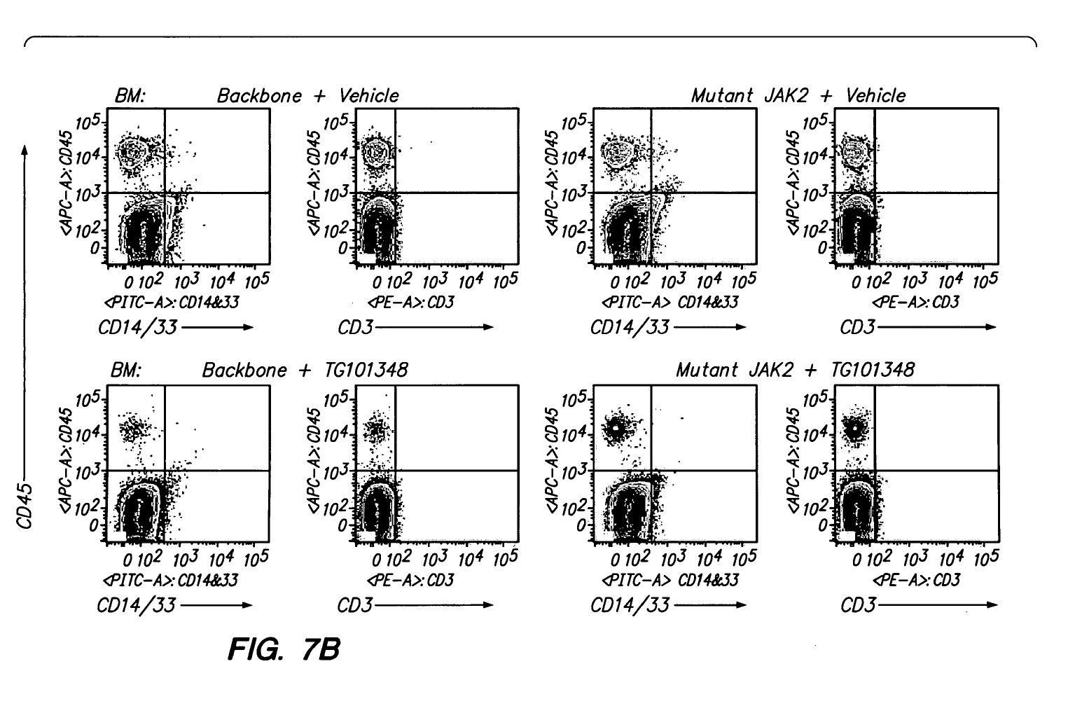

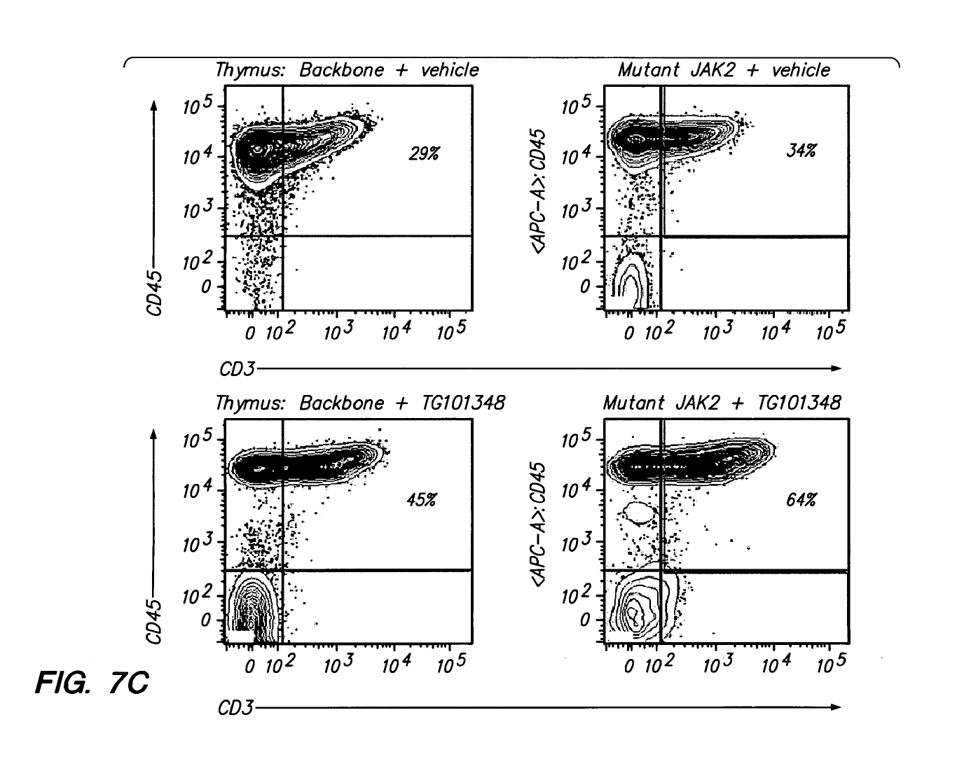

Selective Inhibition of JAK2 V617F-Driven Erythroid Engraftment. To ascertain whether erythroid engraftment was promoted by JAK2 V617F (mutant) or wild-type JAK2 and whether it was susceptible to inhibition with TG101348, normal cord blood progenitors were transduced with backbone, mutant or wild-type JAK2 and transplanted intrahepatically into neonatal RAG2^{-/-}γ_{c}^{-/-} recipients (FIGS. 7A and B). Following 12 days of oral gavage treatment with TG101348, there was a significant inhibition of JAK2 V617F driven erythroid engraftment in transplant recipients while JAK2 wild-type and back bone erythroid engraftment was not significantly reduced (FIGS. 4A and B). These in vivo studies suggested that JAK2 V617F skewed erythroid differentiation potential is particularly sensitive to inhibition by TG101348. Moreover, TG101348 did not inhibit thymic T cell engraftment by mutant JAK2 V617F expressing progenitors suggesting that TG101348 is a highly selective JAK2 and not a JAK3 inhibitor (FIG. 7C).

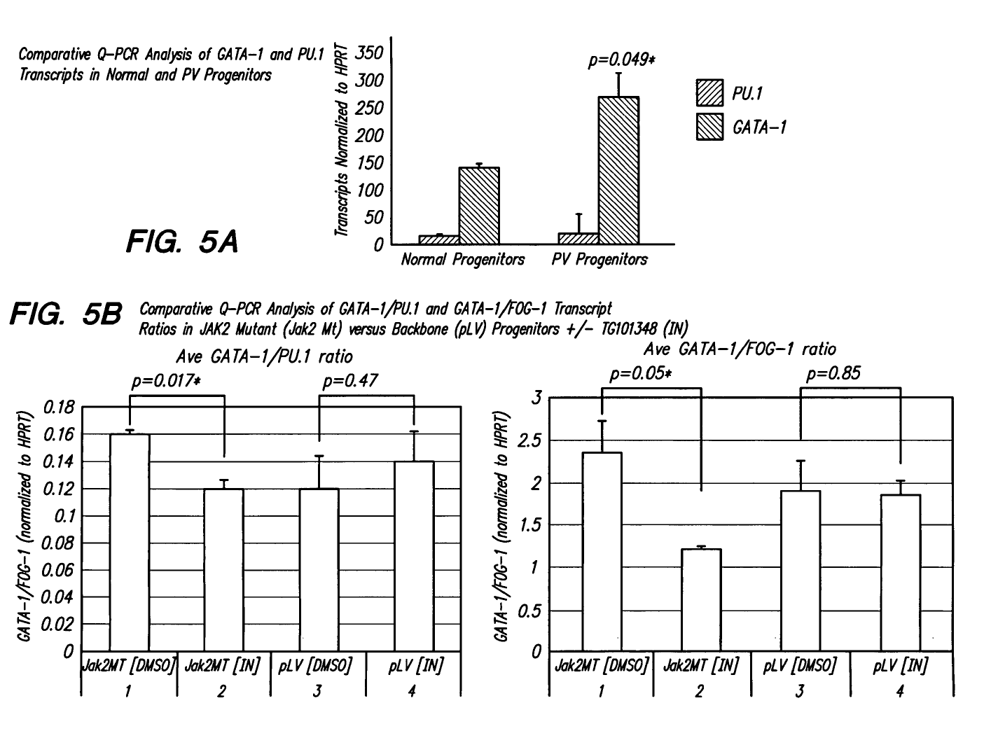

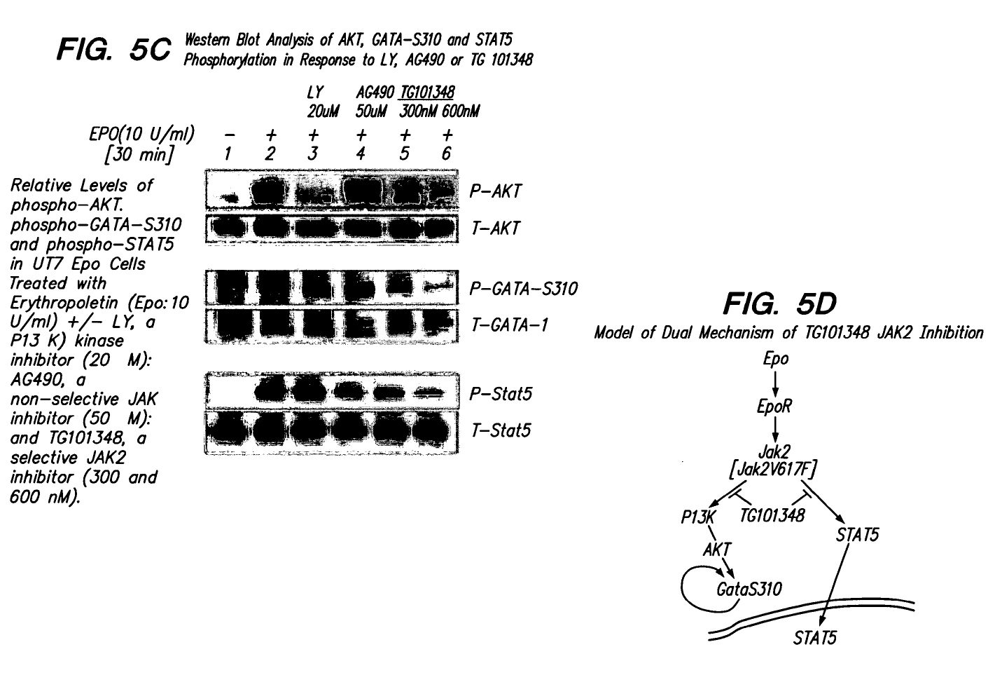

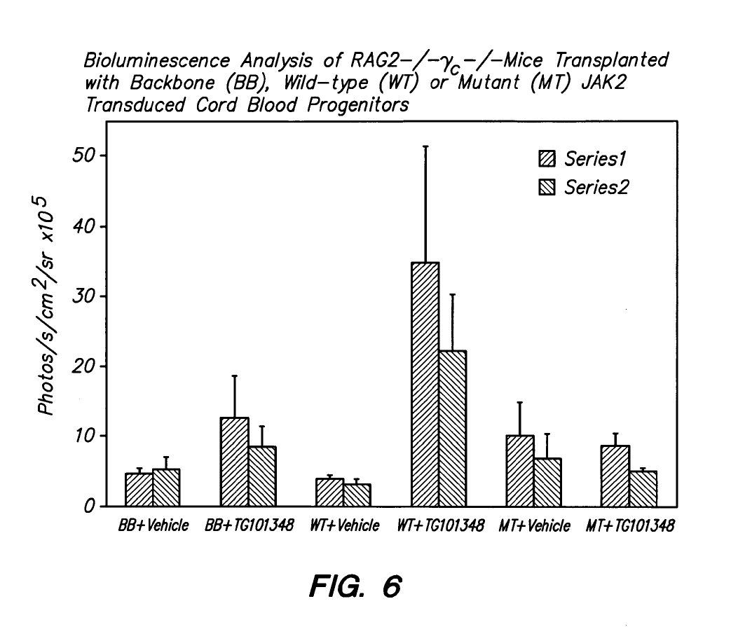

JAK2 Driven Erythroid Signal Transduction Pathways are Inhibited by TG101348. The mechanism of JAK2 V617F enhanced erythroid differentiation was investigated by Q-PCR to detect changes in erythroid (GATA-1), and myeloid (PU.1) transcription factor transcripts in PV progenitors (FIG. 5A). While there were no appreciable differences in PU.1 transcript levels between PV and normal progenitors (p=0.44), there was a significant increase (p=0.049) in GATA-1 expression by PV progenitors in keeping with their enhanced erythroid differentiation potential (FIG. 5A). Similarly, lentiviral transduction of JAK2 V617F enhanced expression of GATA-1 but suppressed expression of FOG-1, a megakaryocytic transcription factor, further skewing the transcriptome profile towards enhanced erythroid differentiation. JAK2 V617F enhancement of GATA-1 in relation to PU.1 transcripts and inhibition of FOG-1 expression was reversed by TG101348 treatment (FIG. 5B).

To better understand the mechanism of TG101348 reversal of enhanced erythroid differentiation, we analyzed the effect of TG101348 on JAK2-mediated signaling in a human erythropoietin responsive cell line UT7-EPO (FIG. 5C). Notably, TG101348 (at 300 and 600 nM) inhibited STAT5 phosphorylation more effectively than AG490, a JAK2 and JAK3 inhibitor. Inhibition of STAT5 phosphorylation was more pronounced with 600 nM TG101348 than with 50 μM or even 100 μM of AG490. While AG490 did not affect AKT phosphorylation in UT7 Epo cells, TG101348 potently inhibited AKT phosphorylation in a manner similar to a PI3 kinase inhibitor, LY294002 (10 μM). Analysis of GATA-1 phosphorylation revealed that addition of erythropoietin enhanced GATA-1 serine 310 (S310) phosphorylation, which was slightly inhibited by LY294002. In contrast, TG101348 reduced GATA-1 S310 phosphorylation, consistent with potent inhibition of AKT phosphorylation (FIG. 5C). Notably, GATA-1 S310 phosphorylation activates GATA-1-mediated transcription of genes involved in erythroid proliferation and differentiation. Thus, inhibition of AKT regulated GATA-1 S310-phosphorylation combined with reduced STAT5 phosphorylation provides a novel dual mechanism for potent and selective inhibition of JAK2 driven erythroid differentiation by TG101348 (FIG. 5D).

Our data revealed an increase in HSC in PV peripheral blood as well as an expansion of the common myeloid progenitor (CMP) pool and emergence of an IL-3 receptor alpha high population during disease progression. In addition, we detected a qualitative alteration in HSC differentiation potential. Moreover, JAK2 V617F mutation expression by PV HSC was clonally transmitted to committed progenitors. Finally, greater inhibition of HSC erythroid differentiation was observed with a JAK inhibitor, AG490, in PV than in normal HSC. These findings suggested that, in addition to enhanced proliferative capacity, one of the main defects in PV may be altered differentiation potential at the stem cell level.

In this study, the role of mutant JAK2 in altering hematopoietic differentiation was demonstrated by lentiviral transduction of cord blood progenitors with JAK2 V617F (mutant JAK2). As seen with JAK2 V617F⁺ PV progenitors, only mutant JAK2 overexpression resulted in enhanced erythroid colony formation, while wild-type JAK2 increased the number of mixed rather than erythroid colonies compared with backbone vector controls. These data suggested a direct link between JAK2 V617F expression and enhanced erythropoiesis in vitro. Furthermore, we demonstrated that the erythroid-skewed differentiation of JAK2 V617F⁺ PV HSC and progenitors was selectively blocked by a potent JAK2 inhibitor, TG101348 (TargeGen Inc), which occupies the ATP binding pocket of JAK2. In addition, TG101348 selectively inhibited erythroid colony formation by JAK2 V617F-transduced cord blood progenitors indicative of an innate sensitivity of mutant JAK2 driven erythroid signal transduction pathways to TG101348 inhibition.

When the in vivo engraftment and differentiation capacity of human PV progenitors was compared with that of normal cord blood progenitors in a bioluminescent xenogenic transplantation model, PV progenitors had similar rates of engraftment but gave rise to increased numbers of human erythroid (glycophorin A⁺) cells in hematopoietic organs of transplanted mice. Targeted inhibition of JAK2 with TG101348 by oral gavage mediated administration resulted in a significant reduction in erythroid engraftment by both JAK2 V617F⁺ PV and mutant JAK2-transduced cord blood progenitors further underscoring the sensitivity of JAK2 V617F-driven erythroid differentiation to TG101348.

Investigation of the mechanisms linking JAK2 V617F expression to enhanced erythroid differentiation in vitro and in vivo by quantitative PCR (Q-PCR), demonstrated that FACS-purified PV stem and progenitor cells harbored higher transcript levels of GATA-1, an erythroid transcription factor, than PU.1, a myeloid transcription factor. This Q-PCR analysis indicated that in PV primitive progenitor cell fate decisions are irrevocably altered by an imbalance in erythroid, GATA-1, versus myeloid, PU.1, transcription factors. To determine whether this imbalance was a consequence of JAK2 V617F expression, we transduced normal cord blood progenitors with mutant JAK2 and analyzed transcript levels of GATA-1, PU.1 and FOG-1, a megakaryocyte transcription factor, by Q-PCR following treatment with vehicle or TG101348 in vitro for 7 days. These experiments revealed that JAK2 V617F enhanced GATA-1 expression was significantly reduced (p=0.013) by TG101348 treatment and that the ratio of GATA-1 to FOG-1 transcripts also decreased significantly (p=0.05) thereby, reestablishing the balance of transcription factors required for normal primitive progenitor differentiation.

Furthermore, Western blot analysis of a human erythropoietin (Epo) responsive cell line demonstrated that TG101348 inhibited activation of GATA-1 regulated transcription by blocking AKT-mediated GATA-1 S310 phosphorylation, potentially providing another mechanism for restoring a balance in the effects of lineage skewing transcription factors. In addition, TG101348 treatment resulted in decreased STAT5 phosphorylation further explaining the potency of TG101348 in blocking JAK2 mediated signaling. Because previous studies involving targeted inhibition of BCR-ABL in CML demonstrated that hematopoietic stem and progenitor cells represent a reservoir for relapse, the capacity of TG101348 to potently inhibit signaling through mutant JAK2 V617F at the primitive progenitor level may be particularly relevant in determining the successful outcome of MPD clinical trials with JAK2 inhibitors.

Recent reports revealed that some MPDs harbor mutations outside the JAK2 V617F region, for example in exon 12 of the JAK2 gene or in the MPL gene. However, over 97% of patients with PV and approximately 50 percent of patients with ET and myelofibrosis harbor the JAK2 V617F mutation, underscoring its importance as a therapeutic target. In this study, inhibition of JAK2 with TG101348 decreased the aberrant erythroid potential of JAK2 V617F positive progenitors both in vitro and in a bioluminescent xenogeneic transplantation model. This occurred in part because of decreased GATA-1 transcription as well as inhibition of both STAT5 and GATA-1 phosphorylation. These data suggest that TG101348, a JAK2 selective tyrosine kinase inhibitor, may be an excellent candidate for targeted therapy of JAK2 driven MPDs.

### Experimental Procedures

Samples. Peripheral blood (n=9) samples, including phlebotomies, were donated on multiple occasions by patients with PV. Normal cord blood (n=8) and peripheral blood samples (n=4) were provided by healthy volunteers. Samples were obtained with informed consent according to Stanford University IRB approved protocols. Normal bone marrow and cord blood samples were also purchased from All Cells™.

Human Hematopoietic Stem Cell and Myeloid Progenitor Flow-Cytometric Analysis and Cell Sorting. Mononuclear fractions were extracted from peripheral blood or bone marrow following Ficoll density centrifugation according to standard methods. Samples were analyzed fresh or subsequent to rapid thawing of samples previously frozen in 90% fetal bovine serum (FBS) and 10% dimethylsulfoxide (DMSO) in liquid nitrogen. In some cases, CD34⁺ cells were enriched from mononuclear fractions with the aid of immunomagnetic beads (CD34⁺ Progenitor Isolation Kit, Miltenyi Biotec, Bergisch-Gladbach, Germany).

Human Hematopoietic Progenitor Assays. Normal and PV HSC(CD34⁺CD38⁻CD90⁺Lin⁻), common myeloid progenitors (CMP; CD34⁺CD38⁺IL-3Ralpha⁺CD45RA⁻Lin⁻), granulocyte-macrophage progenitors (GMP; CD34⁺CD38⁺IL-3Ralpha⁺CD45RA⁺Lin⁻), and megakaryocyte-erythroid progenitors (MEP; CD34⁺CD38⁺IL-3Ralpha⁻CD45RA⁻Lin⁻) were sorted with the aid of a FACS Aria directly into 12 well plates containing complete methylcellulose (GF⁺H4435, StemCell Technologies. Inc., Vancouver, Canada) according to the manufacturer's specifications, with or without 0, 30, 100, 300 or 600 nM of a selective JAK2 inhibitor, TG101348 (TargeGen Inc, San Diego, Calif.). Colonies were incubated in a 37° C. 7% CO₂ humidified incubator and scored on day 14 as colony forming unit-mix (CFU-Mix), burst-forming unit erythroid or colony forming unit-erythroid (BFU-E/CFU-E), CFU-granulocyte (CFU-G), CFU-macrophage (CFU-M), CFU-megakaryocyte (CFU-Mega) or CFU-granulocyte-macrophage (CFU-GM) (Jamieson et al., 2006; Jamieson et al., 2004). Phase contrast photomicrographs of colonies were obtained on day 14 with a Zeiss Axiovert phase-contrast inverted microscope at 50× magnification with the aid of SPOT software.

JAK2 Mutation Screening. JAK2 V617F mutation genotyping was performed on peripheral blood mononuclear cells derived from patients with PV, as well as normal peripheral blood, bone marrow and cord blood. Red blood cells were lysed, and DNA was extracted with the QiaAmp DNA Blood Mini kit according to the manufacturer's directions (Qiagen, Valencia, Calif.) and then stored at −80° C. until amplification-based testing. Extracted DNA was prepared for JAK2 mutation analysis by LightCycler methodology as previously described.

PV Progenitor Colony JAK2 Mutation Analysis. Sequencing analysis of mutant JAK2 expression was performed on pooled PV progenitor colonies treated with vehicle or TG101348. Colonies were plucked and resuspended in 200 μL of RLT buffer supplemented with β-mercaptoethanol (Qiagen RNeasy™) and frozen immediately at −80° C. Samples were thawed and RNA extracted followed by cDNA preparation and PCR amplification with JAK2 specific primers (Jamieson et al., 2006). Mutation analysis of the JAK2 cDNA PCR product was conducted using fluorescent denaturing high performance liquid chromatography (DHPLC) technology and SURVEYOR mismatch cleavage analysis both with the WAVE-HS System (Transgenomic, Gaithersberg, Md.). Aliquots of PCR product (3-15 μl) from all samples were scanned for mutations by DHPLC, confirmed by Surveyor mismatch cleavage, and identified with bidirectional sequence analysis on an ABI 3100 sequencer using BigDye V3.1 terminator chemistry (Applied Biosystems, Inc., Foster City, Calif.). In addition, for semi-quantitative determination of mutant and normal allele frequencies, relative peak areas of DHPLC elution profiles and Surveyor mismatch cleavage products were determined after normalization and comparison to reference controls using the WAVE Navigator software.

Lentiviral JAK2 Vector Construction. Wild-type Jak2 and mutant Jak2V617F (˜3.4 kb) were excised from Jak2-mus-MSCV-neo at the Not I and Age I sites, blunt-ended and cloned into the Sma I site of the self-inactivating lentiviral vector, pLV CMV IRES2 GFP. The pLV CMV IRES2 GFP was modified from pLV CMV GFP Nhe (Naldini et al., 1996) by replacing GFP with IRES2GFP (Clontech). Both WT Jak2 and Jak2V617F sequences were verified using murine Jak2 specific primers flanking the mutation. Lentiviral vectors were simultaneously prepared by co-transfection of WT Jak2, JAK2 V617F or backbone vector, together with pCMV-Δ8.9 and pCMV-VSV-G using Lipofectamine 2000 into 293TFT (Invitrogen, Carlsbad, Calif.). Viral supernatants were collected after 48 h and concentrated by centrifugation. Viral titers of the lentiviral backbone varied in the range of 4×10⁷-1×10⁹ iu/ml.

Progenitor Transduction with Lentiviral Vectors. Wild type JAK2, JAK2 V617F and backbone lentiviral vectors were used to transduce normal cord blood hematopoietic stem cells (HSC) and progenitor cells, which were grown in methocult, with (+) or without (−) TG101348. Colonies were counted at day 14, RNA extracted and reversed transcribed, and amplified with murine JAK2 specific primers. Sequencing of PCR products confirmed the presence of WT JAK2 and JAK2 V617F, respectively.

Bioluminescent Xenogeneic Transplantation Model of Human PV. To assess engraftment potential and in vivo differentiation capacity, JAK2 V617F⁺ PV CD34-enriched cells, HSC or progenitors (CD34⁺CD38⁺Lin⁻) were transduced with lentiviral luciferase GFP (Breckpot et al., 2003) for 48 hours and transplanted intrahepatically into neonatal non-irradiated RAG2^{-/-}γ_{c}^{-/-} mice. Engraftment was analyzed by non-invasive bioluminescent imaging (IVIS 200, Caliper Inc) and by FACS analysis of hematopoietic tissues. In separate experiments, normal cord blood progenitors were transduced with lentiviral luciferase GFP together with JAK2 WT-, MT- and backbone lentiviral vectors followed by intrahepatic transplantation into RAG2−/−γ_{c}−/− mice according to previously published methodology, and analyzed for human engraftment by non-invasive bioluminescent imaging and FACS. Transplanted RAG2^{-/-}γ_{c}^{-/-} mice were also treated with a selective JAK2 inhibitor (TG101348, 300 nM) or vehicle (DMSO) by oral gavage twice daily for 12 days and the effect on engraftment analyzed. In another series of experiments, HSC were transduced with the JAK2 V617F or backbone lentiviral vector with (+) or without (−) TG101348 (IN) or the vehicle (DMSO), grown for 7 days in myelocult media (Stem Cell Technologies Inc) and transcript levels of erythroid transcription factors were quantified by Q-PCR.

Inhibition of EPO signaling in UT7/EPO cells with TG101348. The human erythro-megakaryocytic cell line, UT7/EPO cells were starved in 0.1% FBS in IMDM for 24 h prior to stimulation. Cells were pre-incubated with various inhibitors including LY294002 (10 μM; Calbiochem, San Diego, Calif.), AG490, (50 μM; (Calbiochem, San Diego, Calif.) and TG101348 (300 nM and 600 nM; TargeGen, San Diego, Calif.) 1 h prior to stimulation with 10 U/ml EPO for 30 min. Cells were lysed with 1% NP40 lysis buffer [20 mM Tris-HCl, pH 7.5, 150 mM NaCl, 0.5 mM EDTA, 10% glycerol, complete protease inhibitors and PhosStop (Roche, Indianapolis, Ind.). Equal amounts of lysate (20 μg) were loaded in 4-15% Tris-SDS PAGE gels (Biorad, Hercules, Calif.) and probed with phospho-JAK2 (Tyr1007/1008) (Upstate, Millipore, Billerica, Mass.), phospho-STAT5 (Tyr694) and phospho-AKT (Cell Signaling, Danvers, Mass.), and phospho-GATA-1 (S310) (Abcam, Cambridge, Mass.). Blots were stripped and re-probed with anti-JAK2 (Upstate), pAKT (Cell Signaling), STAT5 (sc-835) (Santa Cruz Biotechnology, Santa Cruz, Calif.) and GATA-1 (Abcam, Cambridge, Mass.).

Quantitative RT-PCR Analysis of GA TA-1, PU.1 and FOG-1 Transcript Levels. Quantitative RT-PCR (Q-PCR) analysis of GATA-1 and PU.1 transcript levels was performed on FACS-purified PV progenitors as previously described. First, HSC and progenitor (1,000 to 50,000) cells were sorted directly into RLT Buffer and total RNA was isolated using RNeasy Micro Kit (Qiagen, Valencia, Calif. USA), according to manufacturer's protocol. Then, a SYBR Greener two-Step Q-RT-PCR Kit for the iCycler (Invitrogen, Carlsbad, Calif., USA) was used to synthesize cDNA and assess GATA-1 and PU.1 relative transcript quantities according to the manufacturer's protocol. Briefly, 8 μl of 4 to 75 ng/μl of RNA were mixed with RT Reaction Mix and RT Enzyme Mix and incubated at 25° C. for 10 min, followed by 50° C. for 30 min and finally 85° C. for 5 min. The tubes were then chilled and 1 μl of RNase H was added to the reaction followed by a 20 min incubation at 37° C. The quantitative PCR (Q-PCR) reaction was performed in duplicate using 2 μl of the template in 25 μl reaction volume containing SYBR Greener Super Mix and 0.4 μM of each forward and reverse primer. Relative values of transcripts were determined according to a standard curve. GATA-1 and PU.1 values were then normalized to HPRT values.

Statistical Analysis. Standard deviation (SD), standard error of the mean (SEM) and numbers of HSC and progenitors per 10⁵ mononuclear cells were measured using FlowJo and Excel software. A two-tailed Student's t-Test (Excel software) was used to analyze statistical differences in the types of colonies derived from PV patient samples treated with vehicle or TG101348 as well as engraftment rates in RAG2^{-/-}γ_{c}^{-/-} mice. A two-tailed t-test (InStat and software) was used to analyze statistical differences in transcript levels of Jak2 V617F transduced CD34 treated with vehicle or TG101348.

FACS Sorting and Analysis. Prior to FACS analysis and sorting, myeloid progenitors were stained with lineage marker specific phycoerythrin (PE)-Cy5-conjugated antibodies including CD2 (RPA-2.10), CD11b (ICRF44), CD20 (2H7), CD56 (B159), GPA (GA-R2) from Becton Dickinson-PharMingen, San Diego, CD3 (S4.1), CD4 (S3.5), CD7 (CD7-6B7), CD8 (3B5), CD10 (5-1B4), CD14 (TUK4), CD19 (SJ25-C1) from Caltag, South San Francisco, Calif. and APCconjugated anti-CD34 (HPCA-2; Becton Dickinson-PharMingen), biotinylated anti-CD38 (HIT2; Caltag) in addition to PE-conjugated anti-IL-3Rα (9F5; Becton Dickinson-PharMingen) and FITC-conjugated anti-CD45RA (MEM56; Caltag) followed by staining with Streptavidin-Alexa 405 to visualize CD38-biotin stained cells and resuspension in propidium iodide to exclude dead cells. Unstained samples and fluorescence-minus one (FMO) controls were included to assess background fluorescence. Following staining, cells were analyzed and sorted using a FACS Aria (Becton Dickinson Immunocytometry Systems, Mountain View, Calif.).

Double sorted hematopoietic stem cells (HSC) were identified as CD34₊CD38⁻CD90₊ and lineage negative. Common myeloid progenitors (CMP) were identified based on CD34₊CD38₊L-3Ra₊CD45RA₋ lin₋ staining and their progeny including granulocyte/macrophage progenitors (GMP) were CD34₊CD38₊IL₋ 3Ra₊CD45RA₊lin₋ while megakaryocyte/erythrocyte progenitors (MEP) were identified based on CD34₊CD38₊IL₋3Ra₋ CD45RA₋lin₋ staining.

Hematopoietic Stem Cell and Progenitor Targeted JAK2 Mutation Analysis. Sample preparation and RNA Extraction. Total RNA was extracted from 190 samples including sorted progenitor cells and colonies using either the RNeasy® Mini Protocol (Qiagen, Germantown, Md.) or TRIZOL® reagent (Invitrogen, Carlsbad, Calif.) according to the instructions of the manufacturers. All samples were quantified using the NanoDrop® ND-1000 Spectrophotometer (Wilmington, Del.) and re-suspended at working concentrations of 50 ng/ul in RNAase free water.

Reverse Transcription and Polymerase Chain Reaction (RT-PCR). RT-PCR amplification of 500 ng of purified RNA was performed with the SuperScript One-Step RT-PCR System with Platinum® Taq (Invitrogen, Carlsbad, Calif.) in individual tubes for each RNA sample, with 1.0 μl of the One-Step RT-PCR Platinum Taq enzyme mixture included in a 2× reaction buffer containing, 0.4 mM of each dNTP, 2.4 mM MgSO₄ and 0.2 μM of the sense and anti-sense gene specific JAK2 primers in a final reaction volume of 25 ul. Reverse transcription and PCR cycling steps were carried out in a MJ Research Dyad thermocycler. The conditions for RT-PCR included cDNA synthesis at 50° C. for 30 min followed by a 2 min denaturing step at 94° C.; and PCR for 35 cycles of denaturation (94° C., 15 sec), annealing (58° C., 30 sec), and extension (68° C., 60 sec) followed by a final extension step of 1 cycle at 68° C. for 5 min.

JAK2 primers used in both the RTPCR and PCR amplifications were: Primary JAK2 primers (forward) 5′-TAAAGGCGTACGAAGAGAAGTAGGAGACT-3′ (reverse) 5′-GGCCCATGCCAACTGTTTAGC-3′. These primers amplify a 301 bp cDNA product that contains JAK2 exon 14, which harbors codon V617. In the event that this One-Step RT-PCR amplification did not yield enough analyzable PCR product, a nested PCR amplification was performed using the product of the One-Step RT-PCR as template in a separate reaction using: Secondary JAK2 primers (forward) 5′-ACGGTCAACTGCATGAAACA-3′ (reverse) 5′-GTTGCTAAACAGTTGGCATGG-3′ These primers amplify a 269 bp cDNA product. Nested PCR was performed using 50 ng of the One-Step RT-PCR product as template in a separate 50 μl reaction which consisted of final concentrations of 1.25 U of HotMaster Taq DNA Polymerase (Eppendorf), HotMaster Taq Buffer with 2.5 mM Mg²⁺ (25 mM Tris-HCL pH 8.0, 35 mM KCL, 0.1 mM EDTA, 1 mM DDT, 50% glycerol, 0.5% Tween20, 0.5% IGEPAL CA-630 and stabilizers), 2 mM of each dNTP, 0.2 μM of each nested sense and anti-sense primer. These primers also served as nested sequencing primers.

Mutation Scanning and DNA Sequencing. Mutation analysis of the JAK2 cDNA PCR product was conducted using fluorescent denaturing high performance liquid chromatography (DHPLC) technology and SURVEYOR mismatch cleavage analysis both with the WAVE-HS System (Transgenomic, Omaha, Nebr.). Aliquots of PCR product (3-15 ┌l) were scanned for mutations by DHPLC, confirmed by Surveyor mismatch cleavage, and identified with bidirectional sequence analysis on an ABI 3100 sequencer using BigDye V3.1 terminator chemistry (Applied Biosystems, Inc., Foster City, Calif.). For semi-quantitative determination of mutant and normal allele frequencies, relative peak areas of DHPLC elution profiles and Surveyor mismatch cleavage products were determined after normalization and comparison to reference controls using the WAVE Navigator software.

Quantitative RT-PCR Analysis of GATA-1, PU.1 and FOG-1 Transcript Levels. In other experiments, RNA was extracted from CD34-enriched cells transduced with mutant Jak2 V617F or lentiviral backbone and treated with TG101348 (IN) or with vehicle (DMSO) (RNEasy MicroKit, Qiagen).₁ In these studies, 150-200 ng of RNA was reversed transcribed using Superscript III for Q-RT-PCR (Invitrogen). Then, cDNA was diluted 1:5 and 15-20 ng was subjected to Q-PCR in a 25 μl reaction mix using 0.4 μM of forward and reverse primers and Sybr GreenER supermix (Invitrogen Corp.) for the iCycler (Biorad, Hercules, Calif.). Q-PCR cycling conditions were 50° C. for 2 min, 95° C. for 8 min 30 s, and 45 cycles of 95° C. 15 s and 60° C. 1 min. Melting curve analysis immediately followed Q-PCR with 95° C. for 1 min, 55° C. for 1 min, and 80 cycles of 55° C.+0.5° C./cycle for 10s. The following primers were used for Q-PCR reactions:

| Gene | Forward Primer | Reverse Primer |
|---|---|---|
| GATA-1 (1) | 5′ actcgaaaccgcaaggcat 3′ | 5′-accaccataaagccaccagct 3′ |
| GATA1 (2) | 5′ cca agc ttc gtg gaa ctc tc 3′ | 5′ cct gtt ctg ccc att cat ct 3′ |
| FOG-1 | 5′ gtc cag acc aga gcc tca tc 3′ | 5′ acc aga gtg cgt cat cct tc 3′ |
| PU.1 | 5′ act cga aac cgc aag gca t 3′ | 5′ acc acc ata aag cca cca gct 3′ |
| HPRT | 5′ agg cgt gca aaa tgg aag g 3′ | 5′ tgg cgt tgg tat aga tcc gtg 3′ |

What is claimed is:

1. A non-human mammal comprising:
exogenous xenogeneic hematopoietic stem and/or progenitor cells; wherein said hematopoietic stem and/or progenitor cells have been genetically altered to express a mutant JAK2 protein.

2. The non-human mammal according to claim 1, wherein said mutant JAK2 protein is human JAK2 V617F and the hematopoietic stem and/or progenitor cells are human cells.

3. The non-human mammal according to claim 2, wherein human cells are transfected with a nucleic acid construct comprising JAK2 V617F, and are introduced into said non-human mammal to provide said hematopoietic stem and/or progenitor cells.

4. The non-human mammal of claim 3, wherein the human cells are cord blood cells.

5. The non-human mammal of claim 4, wherein the cord blood cells are selected for expression of CD34 prior to introduction into said non-human mammal.

6. The non-human mammal of claim 3, wherein said human cells are injected into said non-human animal intrahepatically.

7. The non-human mammal of claim 1, wherein said animal is immunocompromised.

8. The non-human animal of claim 7, wherein said animal is a mouse.

9. The non-human mammal of claim 1, wherein said hematopoietic stem and/or progenitor cells comprise a detectable bioluminescent marker.

10. The non-human mammal of claim 9, wherein the marker is luciferase.

11. The non-human mammal of claim 1, further comprising a malarial pathogen.

12. The non-human mammal of claim 11, further comprising human hepatic tissue.

13. A panel for compound testing, comprising at least 2 mammals according to claim 1, wherein at least one of said mammals comprises a known JAK2 inhibitor, and at least one of said mammals comprises a test compound suspected of erythropoietic modulatory activity.

14. A method for creating a model for human erythropoiesis, the method comprising
transferring by intrahepatic injection a cell population comprising human hematopoietic stem and/or progenitor cells genetically altered to express a mutant JAK2 protein into an immunocompromised non-human mammal host.

15. The method of claim 14, wherein said host is a mouse.

16. A method for screening a candidate therapy for efficacy in treatment of a myeloproliferative disorder associated with JAK2, the method comprising:
transferring by intrahepatic injection a cell population comprising human hematopoietic stem and/or progenitor cells genetically altered to express a mutant JAK2 protein into an immunocompromised non-human mammal host;
wherein said host develops a myeloproliferative disorder;
treating said animals with said candidate therapy;
evaluating the human cells present in said animal.

17. The method of claim 16, further comprises infecting said human cells with a malarial pathogen.


SEQUENCE LISTING (14 sequences)

SEQ ID NO: 1 - DNA, 29 bp, Artificial Sequence
  Primer
taaaggcgtacgaagagaagtaggagact

SEQ ID NO: 2 - DNA, 21 bp, Artificial Sequence
  Primer
ggcccatgccaactgtttagc

SEQ ID NO: 3 - DNA, 20 bp, Artificial Sequence
  Primer
acggtcaactgcatgaaaca

SEQ ID NO: 4 - DNA, 21 bp, Artificial Sequence
  Primer
gttgctaaacagttggcatgg

SEQ ID NO: 5 - DNA, 19 bp, Artificial Sequence
  Primer
actcgaaaccgcaaggcat

SEQ ID NO: 6 - DNA, 21 bp, Artificial Sequence
  Primer
accaccataaagccaccagct

SEQ ID NO: 7 - DNA, 20 bp, Artificial Sequence
  Primer
ccaagcttcgtggaactctc

SEQ ID NO: 8 - DNA, 20 bp, Artificial Sequence
  Primer
cctgttctgcccattcatct

SEQ ID NO: 9 - DNA, 20 bp, Artificial Sequence
  Primer
gtccagaccagagcctcatc

SEQ ID NO: 10 - DNA, 20 bp, Artificial Sequence
  Primer
accagagtgcgtcatccttc

SEQ ID NO: 11 - DNA, 19 bp, Artificial Sequence
  Primer
actcgaaaccgcaaggcat

SEQ ID NO: 12 - DNA, 21 bp, Artificial Sequence
  Primer
accaccataaagccaccagct

SEQ ID NO: 13 - DNA, 19 bp, Artificial Sequence
  Primer
aggcgtgcaaaatggaagg

SEQ ID NO: 14 - DNA, 21 bp, Artificial Sequence
  Primer
tggcgttggtatagatccgtg